(19) United States
(12) Patent Application Publication
(10) Pub. No.: US 2025/0135085 A1
(43) Pub. Date: May 1, 2025

(54) VALVE ASSEMBLIES AND GASKETS FOR OPHTHALMIC SURGICAL CASSETTES

(71) Applicant: Alcon Inc., Fribourg (CH)

(72) Inventors: Behzad HASHEMI, Irvine, CA (US); Ming LU, Placentia, CA (US); Brad Hua CHEN, Mission Viejo, CA (US); Steven Benjamin ROLL, San Clemente, CA (US); Vincent A. BAXTER, Temecula (TW)

(21) Appl. No.: 18/911,793

(22) Filed: Oct. 10, 2024

Related U.S. Application Data
(60) Provisional application No. 63/594,904, filed on Oct. 31, 2023.

(51) Int. Cl. A61M 1/00

(52) U.S. Cl. CPC A61M 1/72; A61M 2205/128

(57) ABSTRACT

Certain embodiments disclosed herein provide a surgical cassette including a housing, one or more retaining rings coupled to the housing, and one or more valve assemblies coupled to the housing by the one or more retaining rings. The one or more valve assemblies configured to control fluid communication between one or more channels within the housing. Each valve assembly includes a valve body having a first end, a second end, a cylindrical surface connecting the first end and second end, and a valve elastomer. The valve elastomer includes a first side configured to couple the valve elastomer to the second end of the valve body and a second side having a recessed surface configured to engage with the housing to form a seal with a base of the housing and reduce mechanical stress across the valve elastomer during rotation of the valve assembly.

### CROSS-REFERENCE TO RELATED APPLICATIONS

This application claims the benefit of U.S. Provisional Application Ser. No. 63/594,904 (filed on Oct. 31, 2023), the content of which is incorporated herein by reference in its entirety.

### INTRODUCTION

Cataract surgery involves removing a cataractous lens and replacing the lens with an artificial intraocular lens (IOL). The cataractous lens is typically removed by fragmenting the lens and aspirating the lens fragments out of the eye. The lens may be fragmented using, e.g., a phacoemulsification probe, a laser probe, or another suitable instrument. During the procedure, the probe fragments the lens, and the fragments are aspirated out of the eye through, e.g., a hollow needle or cannula. Throughout the procedure, irrigating fluid is pumped into the eye to maintain an intraocular pressure (IOP) and prevent collapse of the eye.

During cataract surgery, a surgical cassette having one or more peristaltic and/or venturi pumps and one or more valve assemblies may be operably coupled with a fluidics module of a surgical console and used to facilitate the aspiration and irrigation functionalities described above. In general, the one or more valve assemblies of the surgical cassette are operable to control the application of flow generated by the one or more peristaltic pumps during the surgical procedure.

However, conventional surgical cassettes have a number of significant shortcomings including, for example, relatively tight sealing tolerances, relatively high torque required for valve operation, restricted flow capacity, the inability to maintain a secure connection with the surgical console, relatively high fabrication costs and complexity, and redundancy issues, among others.

### BRIEF SUMMARY

The present disclosure relates generally to ophthalmic surgical cassettes, valve assemblies therefor, and methods of use thereof.

In certain embodiments, a surgical cassette is provided which is configured to be coupled to a surgical console for ophthalmic irrigation, infusion, suction, and/or aspiration during a surgical procedure. The surgical cassette includes a housing having a partition separating a first surface of the housing from a second surface of the housing, one or more ports formed in the first surface of the housing, and one or more corresponding channels adjoining the second surface of the housing and in fluid communication with the one or more ports. The surgical cassette includes one or more valve assemblies coupled to the housing and configured to control fluid communication between the one or more channels of the housing. Each of the one or more valve assemblies includes a valve body having a first end, a second end, a cylindrical surface connecting the first end and second end, and a valve elastomer. The valve elastomer includes a first side configured to couple the valve elastomer to the second end of the valve body. The valve elastomer also includes a second side having a recessed surface configured to engage with the housing to form a seal with a base of the housing, and to reduce mechanical stress across the valve elastomer during rotation of the valve assembly. The valve body is rotatable about a first axis orthogonal to the first end and relative to the first surface of the housing to align one or more passages with the one or more ports of the housing to open fluid communication between the one or more corresponding channels. A drive interface is formed on the first end of the valve body and configured to engage a drive mechanism for rotating the valve body.

In certain embodiments, a surgical cassette is provided which is configured to be coupled to a surgical console for ophthalmic irrigation, infusion, suction, and/or aspiration during a surgical procedure. The surgical cassette includes a housing having a partition separating a first surface of the housing from a second surface of the housing, one or more ports formed in the first surface of the housing, and one or more channels adjoining the second surface of the housing and in fluid communication with the one or more ports. The surgical cassette includes one or more valve assemblies coupled to the housing and configured to control fluid communication between the one or more channels of the housing. Each of the one or more valve assemblies includes a valve body having a first end, a second end, and a cylindrical surface connecting the first end and second end. The valve body is rotatable about a first axis orthogonal to the first end and relative to the first surface of the housing to align one or more passages with one or more of the one or more ports of the housing to open fluid communication between the one or more corresponding channels. A drive interface is formed on the first end of the valve body and configured to engage a drive mechanism for rotating the valve body. The surgical cassette includes one or more valve gaskets configured to form a seal between the one or more valve assemblies and the housing. Each valve gasket includes an inner portion comprised of a first material and an outer portion comprised of a second material. The outer portion surrounds the inner portion and is coupled to the housing.

The following description and the related drawings set forth in detail certain illustrative features of one or more embodiments.

### BRIEF DESCRIPTION OF THE DRAWINGS

The appended figures depict certain aspects of one or more disclosed embodiments and are therefore not to be considered limiting of the scope of this disclosure.

FIG. 1A illustrates an example of an ophthalmic surgical system that may be used to perform ophthalmic procedures on an eye, according to certain embodiments.

FIG. 1B is an example of subsystems of a console of the ophthalmic surgical system of FIG. 1A, according to certain embodiments.

FIG. 2A is a back side isometric view of an example surgical cassette which may be operably coupled to a console of an ophthalmic surgical system, according to certain embodiments.

FIG. 2B is a back side elevation view of the surgical cassette of FIG. 2A, according to certain embodiments.

FIG. 2C is an exploded front side isometric view of the surgical cassette of FIG. 2A, according to certain embodiments.

FIG. 2D is an exploded back side isometric view of the surgical cassette of FIG. 2A, according to certain embodiments.

FIG. 2E is a front side elevation view of a base of the surgical cassette of FIG. 2A, according to certain embodiments.

FIGS. 2F-2G are enlarged exploded front side and back side isometric views, respectively, of a portion of the surgical cassette of FIG. 2A illustrating an example valve assembly having two passages in the valve body, according to certain embodiments.

FIG. 3A is a top side isometric view illustrating an example valve body having two passages, according to certain embodiments.

FIG. 3B is a bottom side isometric view illustrating the valve body having two passages seen in FIG. 3A, according to certain embodiments.

FIG. 3C is a cross sectional side view illustrating the valve body having two passages seen in FIG. 3A, according to certain embodiments.

FIG. 3D is a top down perspective view illustrating the valve body having two passages seen in FIG. 3A, according to certain embodiments.

FIG. 4A is a top side isometric view illustrating another example valve body having only one passage, according to certain embodiments.

FIG. 4B is a bottom side isometric view illustrating the valve body having only one passage seen in FIG. 4A, according to certain embodiments.

FIG. 4C is a cross sectional side view illustrating the valve body having only one passage seen in FIG. 4A, according to certain embodiments.

FIG. 4D is a top down perspective view illustrating the valve body having only one passage seen in FIG. 4A, according to certain embodiments.

FIG. 5A is a cross sectional side view illustrating the valve body seen in FIGS. 3A or 4A, disposed in the surgical cassette of FIG. 2A, according to certain embodiments.

FIG. 5B is a magnified view of FIG. 5A illustrating a cross sectional top portion of the valve body disposed in the surgical cassette, according to certain embodiments.

FIGS. 6A-6B are front side elevation views of a portion of the surgical cassette of FIG. 2A illustrating two different valve positions, according to certain embodiments.

FIG. 7A is an enlarged isometric view of a portion of an alternative embodiment of base and housing of FIGS. 2A-2G, according to certain embodiments.

FIG. 7B is a cross sectional side view of the portion of the base and housing in FIG. 7A illustrating a port, a protruding ring, and the base, according to certain embodiments.

FIG. 7C is a top perspective view of the portion of the base and housing in FIG. 7A illustrating ports, protruding rings, and the base, according to certain embodiments.

FIG. 8A is an enlarged isometric view of a portion of an alternative embodiment of base and housing of FIGS. 7A-7C, according to certain embodiments.

FIG. 8B is a cross sectional side view of the portion of the base and housing in FIG. 8A illustrating a recessed surface and the base, according to certain embodiments.

FIG. 8C is a top perspective view of the portion of the base and housing in FIG. 8A illustrating the recessed surface and the base, according to certain embodiments.

FIG. 9 is an enlarged isometric view of a portion of an alternative embodiment of base and housing of FIGS. 7A-7C and 8A-8C, according to certain embodiments.

FIG. 10A is an exploded back side isometric view illustrating an alternative valve assembly, according to certain embodiments.

FIG. 10B is an isometric front side view of the valve gasket of FIG. 10A, according to certain embodiments.

FIG. 10C is a cross sectional side view illustrating the valve gasket and valve assembly seen in FIG. 10A, according to certain embodiments.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

The present disclosure relates generally to ophthalmic surgical cassettes, valve assemblies therefor, and methods of use thereof.

Certain embodiments disclosed herein provide valve assemblies for a surgical cassette with improved sealing. For example, certain embodiments herein provide valve elastomers having a recessed surface between planar surfaces. In certain embodiments, a valve body has a planar end surface perpendicular to a cylindrical outer surface of the valve body which seals with a corresponding surface of the cassette housing. Because the sealing interface is on a longitudinal end of the valve body, this arrangement may be referred to herein as “end-sealing.”

Certain embodiments disclosed herein provide lower fabrication cost and complexity when compared to conventional valves. For example, certain embodiments disclosed herein eliminate the need for high ultrasonic weld joint strength in conventional valve and cassette bodies, lower reaction force of valve body and valve assemblies, and provide adequate sealing for preventing liquid from travelling between ports. In addition, in certain embodiments disclosed herein, valve assemblies are more easily molded and assembled (e.g., ultrasonically welded, bonded, or snap fit) when compared to conventional valves.

Certain embodiments disclosed herein provide greater flexibility in the layout of the surgical cassette including, e.g., the arrangement of valve assemblies and flow channels therein. For example, when compared to conventional surgical cassettes, certain embodiments disclosed herein provide greater flexibility on tolerance stack-up, prevent changes in sealing force over time, and improve duration in shelf-life.

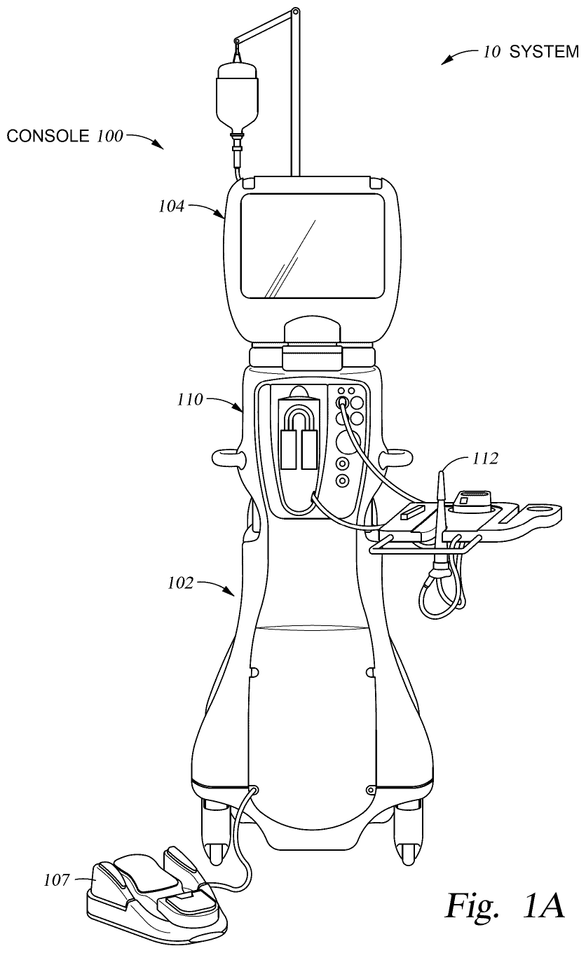

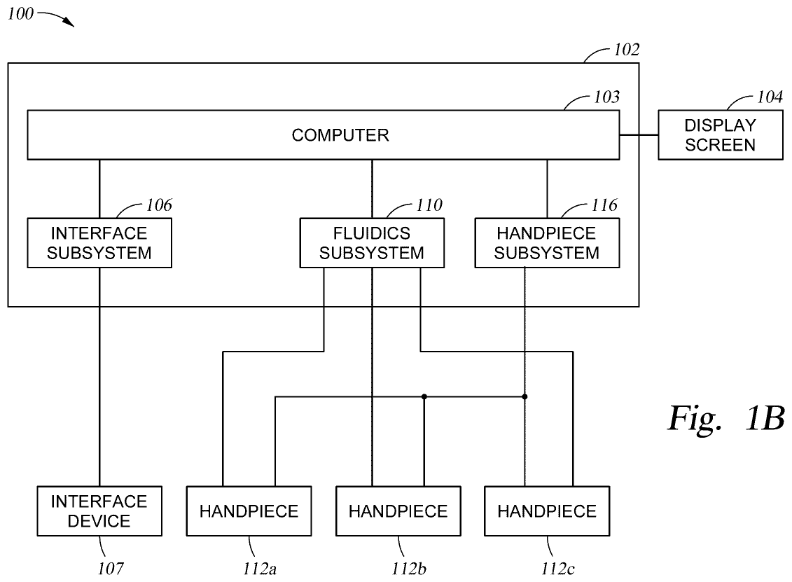

FIG. 1A illustrates an example of an ophthalmic surgical system 10 that may be used to perform ophthalmic procedures on an eye, according to certain embodiments. In the illustrated embodiments, system 10 includes console 100 (also referred to as a “surgical console”), an interface device 107 (e.g., a foot pedal), and a handpiece 112. Console 100 includes a housing 102, a display screen 104, and a fluidics subsystem 110. The components of system 10 and console 100 may be coupled as shown and described in more detail with reference to FIG. 1B.

FIG. 1B illustrates example subsystems of console 100 of ophthalmic surgical system 10 of FIG. 1A, according to certain embodiments. Console 100 includes housing 102, which accommodates a computer 103 (with an associated display screen 104) and subsystems 106, 110, and 116, which support interface device 107 and handpieces 112 (112a-c). An interface device 107 receives input to console 100, sends output from console 100, and/or processes the input and/or output. Examples of an interface device 107 include a foot pedal, manual input device (e.g., a keyboard), and a display. Interface subsystem 106 receives input from and/or sends output to interface device 107.

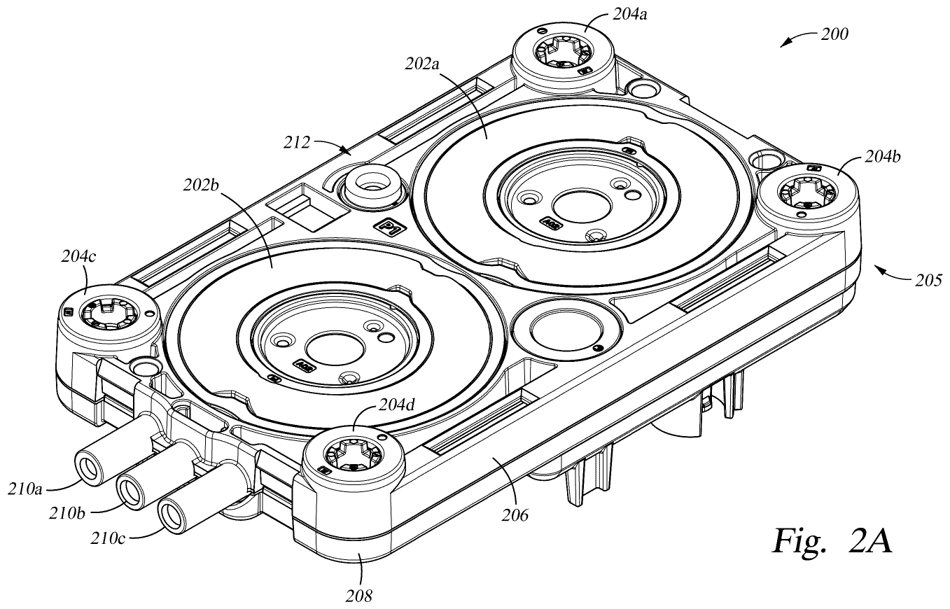

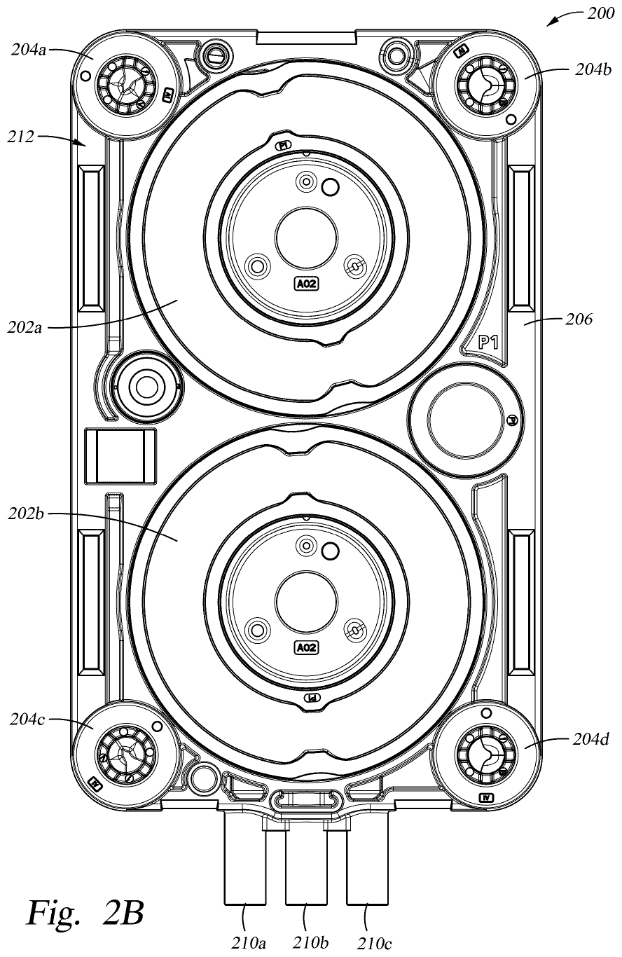

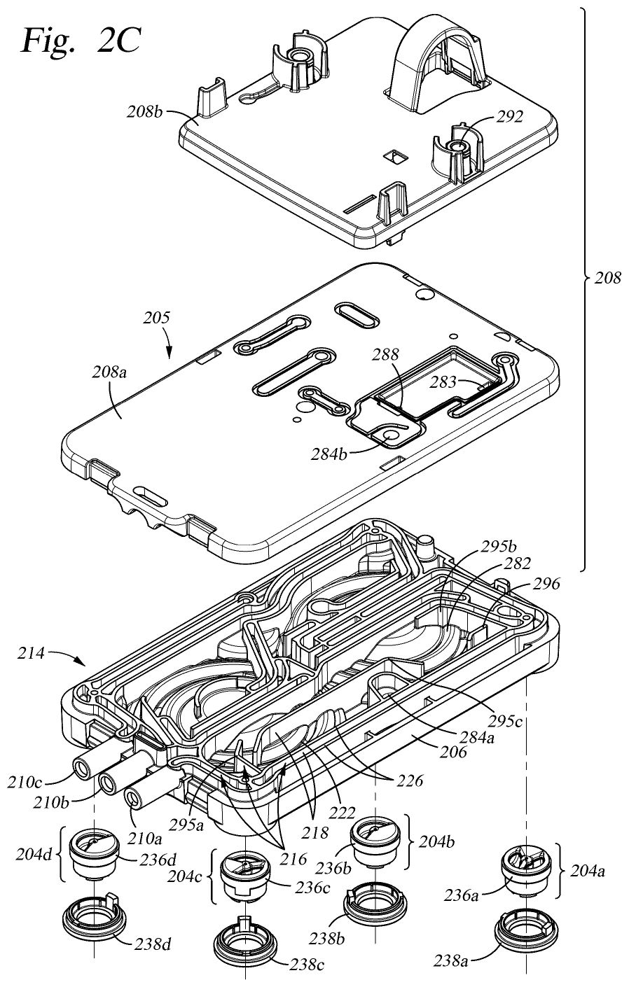

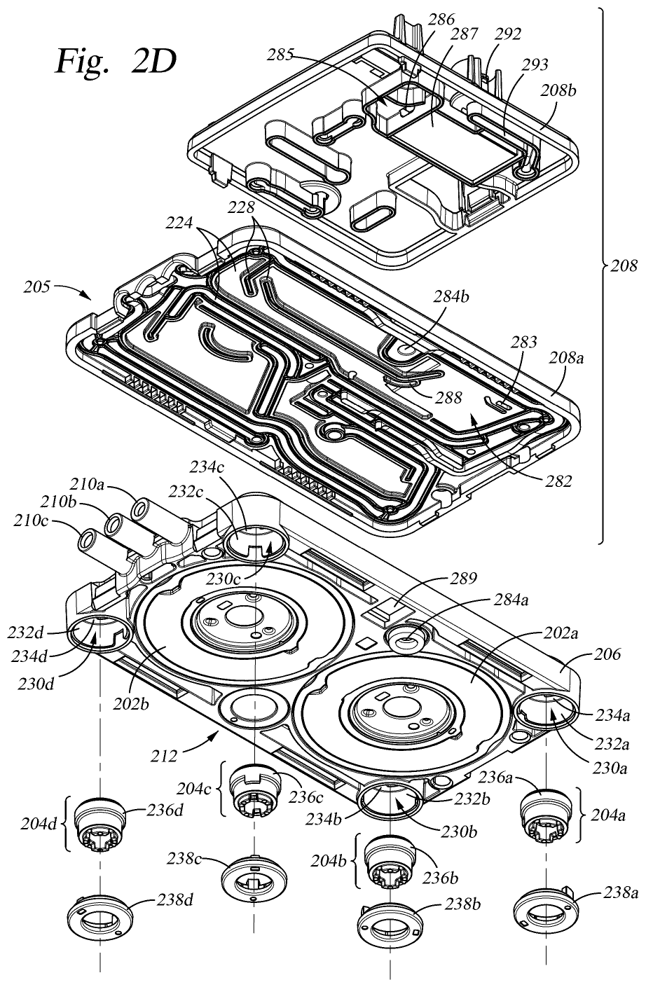

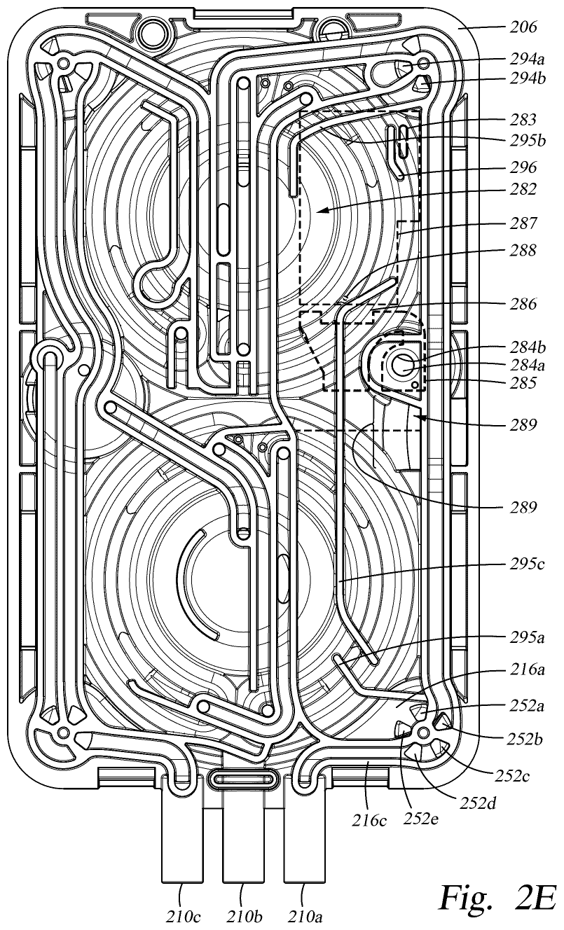

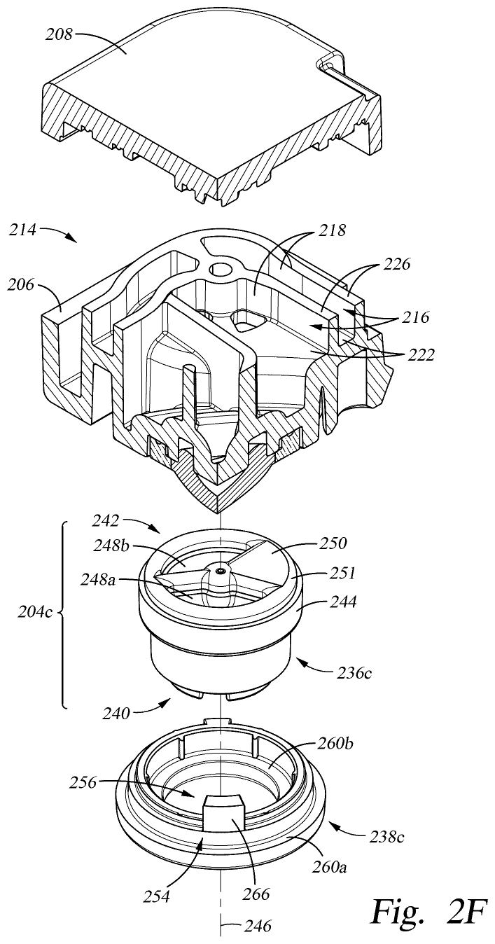

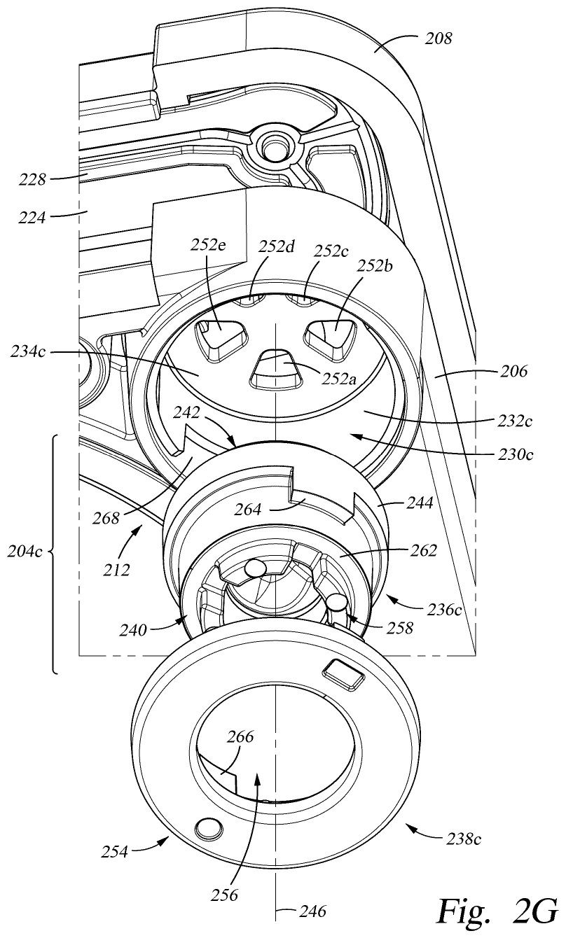

Fluidics subsystem 110 provides fluid control for one or more handpieces 112 (112a-c). For example, fluidics subsystem 110 may manage fluid for an irrigating cannula. In some embodiments, fluidics subsystem 110 may be operatively coupled to a surgical cassette (e.g., surgical cassette 200 of FIGS. 2A-2B) during a surgical procedure. For example, surgical cassette 200 may be inserted into, attached to, and/or integrated with fluidics subsystem 110 via a coupling mechanism. The coupling mechanism may comprise one or more of a latching mechanism, locking mechanism, or other similar connection mechanism. When fluidics subsystem 110 is operatively coupled to surgical cassette 200, fluidics subsystem 110 may control irrigation and/or aspiration of fluids through surgical cassette 200. In certain embodiments, the fluidics subsystem 110 includes one or more mechanical pumps having roller pump heads configured to engage with one or more corresponding pump assemblies on the surgical cassette 200, described below. The engagement of the roller pump heads and pump assemblies generates a source of pressure and/or vacuum utilized during an ophthalmic surgical procedure.

Handpiece 112 may be any suitable ophthalmic surgical instrument, e.g., an ultrasonically-driven phacoemulsification (phaco) handpiece, a laser handpiece, an irrigating cannula, a vitrectomy handpiece, or another suitable surgical handpiece.

Handpiece subsystem 116 supports one or more handpieces 112. For example, handpiece subsystem 116 may manage ultrasonic oscillation for a phaco handpiece, provide laser energy to a laser handpiece, control operation of an irrigating cannula, and/or manage features of a vitrectomy handpiece.

Computer 103 controls operation of ophthalmic surgical system 10. In certain embodiments, computer 103 includes a controller that sends instructions to components of system 10 to control system 10. A display screen 104 shows data provided by computer 103.

FIG. 2A is a back side isometric view of an example surgical cassette 200 which may be operably coupled to a console of an ophthalmic surgical system (e.g., console 100 of ophthalmic surgical system 10 illustrated in FIGS. 1A-1B), according to certain embodiments. For example, as described above, surgical cassette 200 may be operatively coupled to fluidics subsystem 110 of console 100. FIG. 2B is a back side elevation view of surgical cassette 200 of FIG. 2A, according to certain embodiments. FIGS. 2A-2B are described together herein for clarity.

In some embodiments, the general operations of the surgical cassette 200 may be described in more detail in U.S. Patent Application No. 63/175,589 filed on Apr. 16, 2021, entitled “Systems and Methods for Post-Occlusion Break Surge Mitigation”, which is hereby incorporated by reference in its entirety (other manners of surgical cassette operation are also contemplated). Surgical cassette 200 includes two pump assemblies 202 (202a-b) which provide a source of pressure and/or vacuum and four valve assemblies 204 (204a-d) which control pressure and/or fluid communication within surgical cassette 200. In certain other embodiments, there may be only one pump assembly or more than two pump assemblies. In certain other embodiments, there may be more or less than four valve assemblies (e.g., 2-6 valve assemblies).

In certain embodiments, an external source of pressure and/or vacuum is coupled to surgical cassette 200. In such embodiments, the external source may be either in place of or in addition to pump assemblies 202.

Surgical cassette 200 has a housing 205 including a base 206, a cover assembly 208 coupled to base 206, and inlet/outlet ports 210 (210a-c) in base 206 which provide pressure and/or fluid communication between inside and outside of the housing 205. Flow lines (e.g., tubing) may also be coupled between each port 210a-c and a corresponding component of fluidics subsystem 110 and/or a corresponding handpiece 112a-c (shown in FIGS. 1A-1B).

In certain embodiments, one of a first pump assembly 202a or second pump assembly 202b provides a source of pressure (e.g., to create a driving force for fluid irrigation) while the other one of the first pump assembly 202a or second pump assembly 202b provides a source of vacuum (e.g., to create suction for fluid aspiration). The first pump assembly 202a and second pump assembly 202b may be peristaltic pumps or any other suitable type of pump for generating pressure and/or vacuum. In certain embodiments, the first pump assembly 202a and second pump 202b assembly are identical to each other.

Valve assemblies 204 are coupled to base 206. Valve assemblies 204 function cooperatively to control pressure and/or fluid communication within and through surgical cassette 200. In the illustrated embodiments, surgical cassette 200 includes a first valve assembly 204a, a second valve assembly 204b, a third valve assembly 204c, and a fourth valve assembly 204d. As shown, in the embodiments of FIG. 2A, the four valve assemblies 204 are arranged at the four corners of housing 205 and surrounding the two pump assemblies 202 which are arranged towards a center of housing 205. However, in certain other embodiments, pump assemblies 202 and valve assemblies 204 may have any other suitable arrangement.

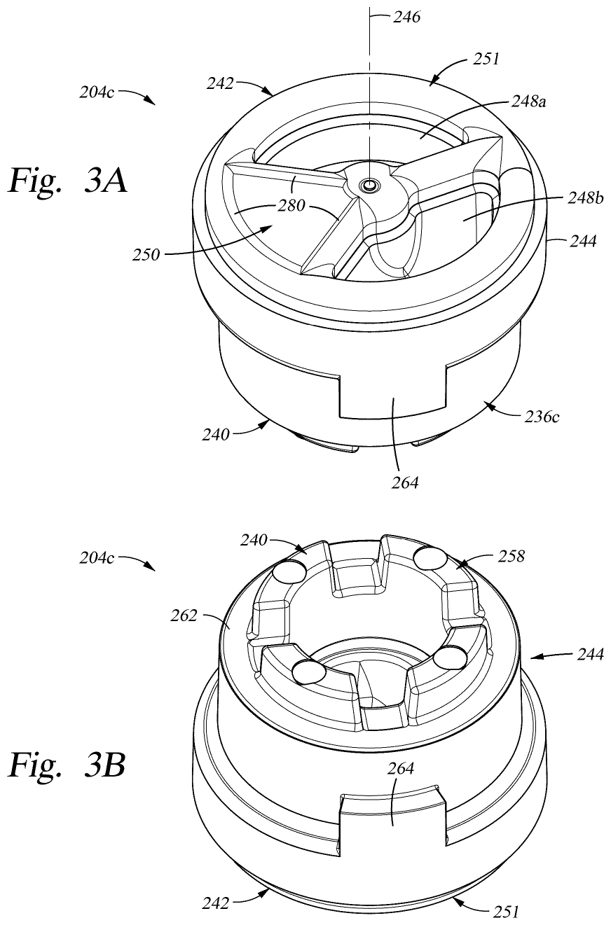

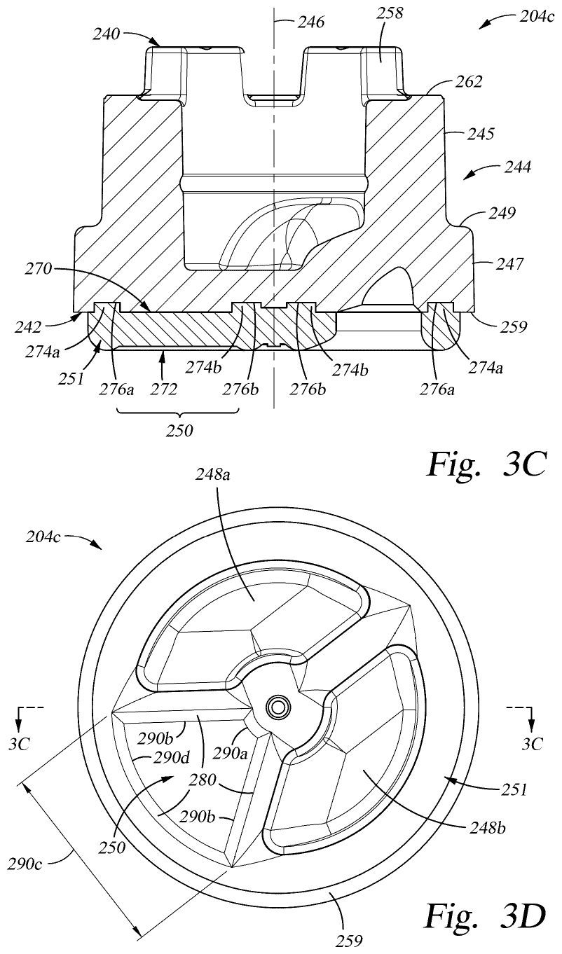

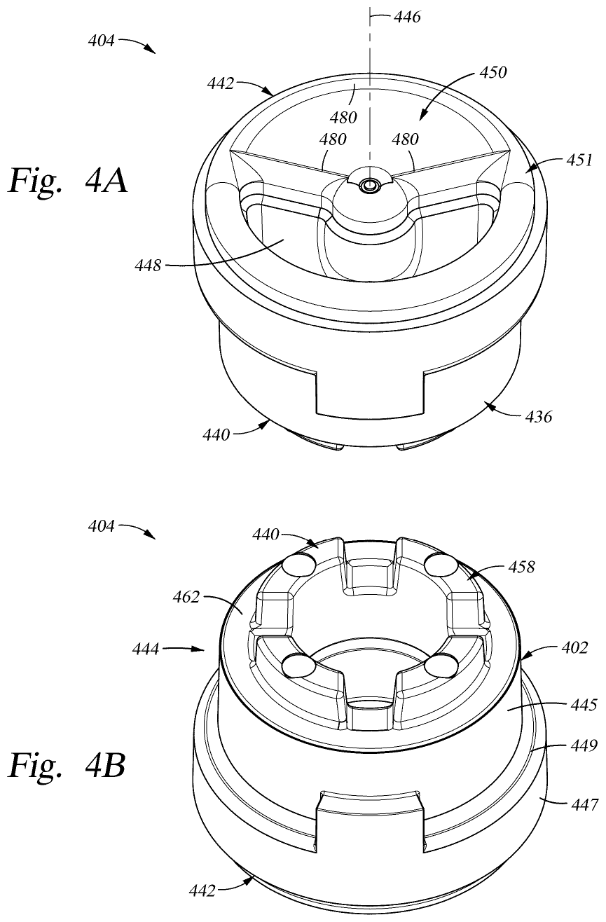

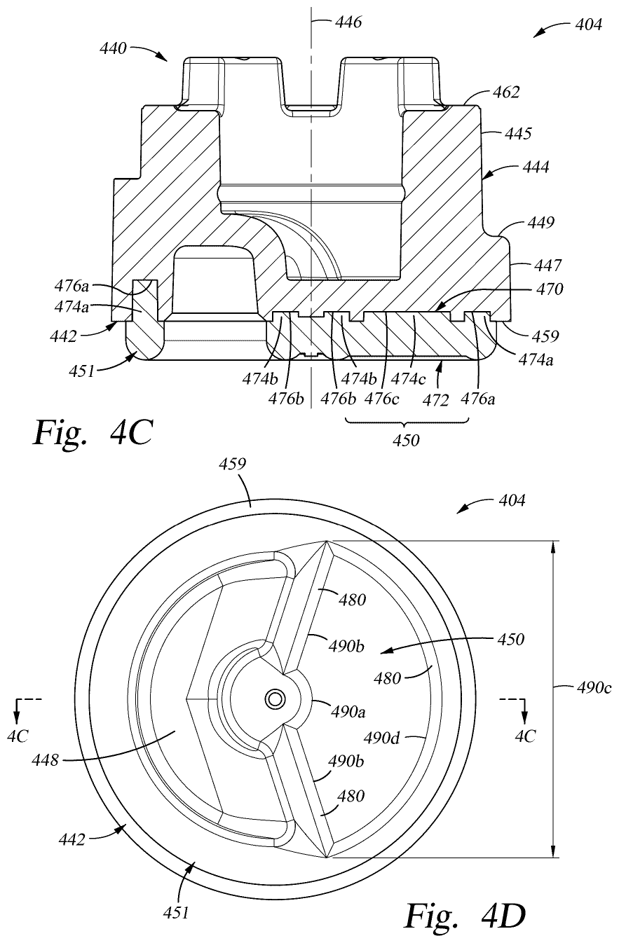

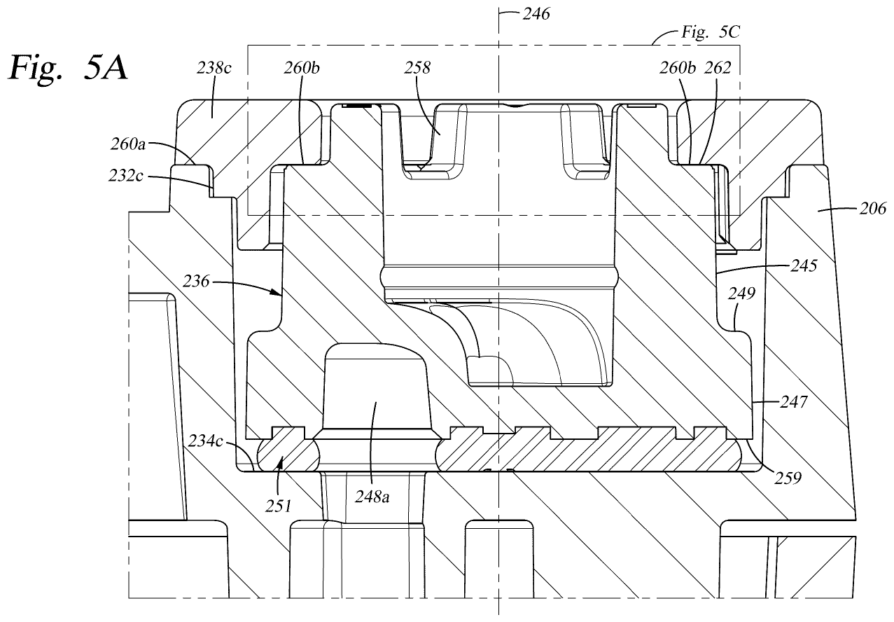

In certain embodiments, valve assemblies 204 may be operated to route fluid flow selectively between one or more channels of housing 205 as described in more detail below with respect to FIGS. 3A-3B. Alternative valve assemblies which may be implemented in surgical cassette 200 are described in more detail below with reference to FIGS. 4A-4B.

In some embodiments, first valve assembly 204a and third valve assembly 204c may be in pressure and/or fluid communication with first pump assembly 202a and port 210a to provide aspiration (suction) through port 210a during an operation, and second valve assembly 204b and fourth valve assembly 204d may be in pressure and/or fluid communication with second pump assembly 202b and port 210c to provide irrigation (infusion) through port 210c during the same operation.

Pump assemblies 202 and valve assemblies 204 are located on a back side 212 of base 206 which is visible in FIGS. 2A-2B. Cover assembly 208 is coupled to a front side 214 (shown in FIG. 2C) of base 206 which faces away from back side 212. In certain embodiments, cover assembly 208 may be welded, bonded, or fastened to base 206 using any suitable coupling mechanism. For example, cover assembly 208 may be coupled to base 206 using a solid-state welding technique (e.g., ultrasonic welding in which high-frequency ultrasonic acoustic vibrations are locally applied to work parts being held together under pressure to create a solid-state weld).

Back side 212 of base 206 is configured to interface with console 100 when surgical cassette 200 is coupled thereto. For example, a drive interface on a valve body of each valve assembly 204 may engage a corresponding drive mechanism of console 100 for rotating the corresponding valve body. The valve body is described in more detail below with respect to FIGS. 2F-2G. In certain embodiments, the drive mechanism is a direct drive motor which operates at lower torque with faster valve response time when compared to a geared drive motor which is conventionally used. However, the embodiments described herein may use any suitable type of drive motor. The components of surgical cassette 200 are described in more detail below with respect to FIGS. 2C-2D.

FIG. 2C is an exploded front side isometric view of surgical cassette 200 of FIG. 2A, according to certain embodiments. FIG. 2D is an exploded back side isometric view of surgical cassette 200 of FIG. 2A, according to certain embodiments. FIGS. 2C-2D are described together herein for clarity. In the illustrated embodiments, cover assembly 208 includes two separate pieces including first cover piece 208a (also referred to as a “cover”) coupled directly to front side 214 of base 206 and second cover piece 208b (also referred to as a “handle”) coupled to cover 208a. In some other embodiments, cover assembly 208 may consist of only a single piece.

A plurality of channels 216 are formed in housing 205. The plurality of channels 216 are arranged to provide a plurality of independent fluid paths between pump assemblies 202, valve assemblies 204, and inlet/outlet ports 210. Each channel 216 is longitudinally defined in a first direction parallel to a plane of housing 205. Each channel 216 includes sidewalls 218 oriented perpendicular to the first direction which enclose the corresponding channel 216 therebetween. In addition, a depth of each channel 216 is defined in a second direction perpendicular to the first direction between a lower wall (also referred to as a “partition”) of base 206 (e.g., front side surface 222 thereof) and an inner surface 224 (shown in FIG. 2D) of cover 208a.

The plurality of channels 216 are sealingly enclosed through contact between a first surface 226 formed on front side 214 of base 206 and a corresponding face 228 (shown in FIG. 2D) extending from inner surface 224 of cover 208a. In order to effect sealing, first surface 226 and corresponding face 228 are molded to include matching contours and assembled to ensure precise alignment between opposing sides. In some other embodiments, the plurality of channels 216 may be formed in cover 208a instead of base 206 and sealingly enclosed through contact between a back side surface of cover 208a and a corresponding front side face of base 206. In some other embodiments, base 206 and cover 208a may be integrally formed as a single piece. In such embodiments, base 206 and cover 208a may be injection molded with the use of a slide technique.

Four bores 230 (230a-d) are formed in back side 212 of base 206 for receiving a corresponding valve assembly 204. Each bore 230 is defined by a cylindrical inner wall 232 (232a-d) and a first side 234 (234a-d). In certain other embodiments, there may be more or less than four bores to correspond to each valve assembly. One or more ports (e.g., five shown in FIGS. 2F-2G) are formed through base 206. The ports connect front side surface 222 and opposite the first side 234 of base 206. Each port corresponds to one of the plurality of channels 216 in contact with, or adjoining, front side surface 222 of base 206. The ports are described in more detail below with respect to FIGS. 2F-2G.

Each valve assembly 204 generally includes a valve body 236 (236a-d) and a valve elastomer 251 that is configured to be disposed in a corresponding bore 230. Valve body 236 is coupled to the housing by a retaining ring 238 (238a-d), such that valve body 236 and retaining ring 238 fit together in a stacked arrangement. Valve body 236 is disposed between a first side 234 of base 206 and corresponding retaining ring 238. Retaining ring 238 applies a sealing force on corresponding valve body 236 to press valve body 236 against first side 234 of base 206 as described in more detail below. In some other embodiments, instead of being defined within base 206, each bore 230 may be defined within a corresponding retaining ring 238 that fits around a corresponding valve body 236. In some other embodiments, each valve body 236 may be rotatably coupled to base 206 using a retention cap. The retention cap may be disposed through valve body 236 (e.g., aligned with a longitudinal axis of valve body 236). In such embodiments, each valve body 236 may be coupled to base 206 without being disposed in a corresponding bore 230.

A venturi reservoir 282 is disposed inside housing 205 of surgical cassette 200 between base 206 and cover 208a. Venturi reservoir 282 serves at least two primary functions that are integral to the operation of surgical cassette 200. In general, venturi reservoir 282 provides a vacuum source for suction of fluids during a venturi operation and provides a fluid volume sink for venting vacuum pressure that may build-up within surgical cassette 200 in the event of a post-occlusion break surge.

FIG. 2E is a front side elevation view of base 206, according to certain embodiments. Note that in FIG. 2E, some parts of cover 208a and handle 208b are shown in phantom for illustrative purposes. Although some aspects of venturi reservoir 282 are visible in FIGS. 2C-2D, certain aspects are illustrated more clearly in FIG. 2E. FIGS. 2C-2E are, therefore, described together herein for clarity.

Surgical cassette 200 may be coupled to an external vacuum source (e.g., a venturi source) disposed in console 100. Vacuum pressure from the external vacuum source is applied to venturi reservoir 282 through a vacuum port 283 in cover 208a. In the illustrated embodiments, vacuum port 283 is an elongated slot. In certain embodiments, vacuum pressure within venturi reservoir 282 is about 720 mmHg (millimeters of mercury). In certain embodiments, maximum air flow through vacuum port 283 is about 1.2 standard liters per minute. The external vacuum source is configured to apply vacuum pressure to venturi reservoir 282 through a vacuum flow path that passes from upstream to downstream through vacuum port 283 in cover 208a and into handle 208b where the air is filtered before passing back through respective openings in cover 208a and base 206 before reaching the external vacuum source. The vacuum flow path is described in more detail below.

In certain embodiments, an opening 284a in back side 212 of base 206 is coupled to a vacuum port on console 100 leading to the external vacuum source. Opening 284b in cover 208a is aligned with opening 284a in base 206. Openings 284a-b are oriented in a direction perpendicular to a plane of base 206. Openings 284a-b are in pressure communication between the external vacuum source and a catch reservoir 285 that is defined between cover 208a and handle 208b. Catch reservoir 285 is in fluid communication, through opening 286, with a filter 287 that is disposed between cover 208a and handle 208b. Catch reservoir 285 catches liquid that leaks through filter 287 and prevents the leaked liquid from entering the vacuum source.

Filter 287 is in pressure communication between the external vacuum source and vacuum port 283 in cover 208a. The location of filter 287 overlays at least a portion of venturi reservoir 282 enabling the use of a more compact housing 205 compared to other designs. An upstream side of filter 287 faces towards cover 208a. A downstream side of filter 287 faces towards handle 208b. Filter 287 seals with handle 208b to prevent liquid from leaking around filter 287. Filter 287 permits air to pass from vacuum port 283 to catch reservoir 285 in handle 208b while blocking liquid from passing to the downstream side of filter 287 and into the external vacuum source. In certain embodiments, filter 287 is directly coupled to handle 208b (e.g., ultrasonically welded). In certain embodiments, filter 287 is hydrophobic, and thus impermeable to aqueous fluids.

In certain cases, liquid is able to pass through vacuum port 283 and becomes trapped between cover 208a and the upstream side of filter 287. A filter drain hole 288 is disposed through cover 208a in a position overlaying a lower end of filter 287. Filter drain hole 288 is in fluid communication between the upstream side of filter 287 and venturi reservoir 282 to drain the trapped liquid back into venturi reservoir 282. Draining of the trapped liquid through filter drain hole 288 helps maintain maximum usable filter area for maximum air flow through vacuum port 283. In certain embodiments, filter drain hole 288 is much smaller than vacuum port 283 in terms of cross-sectional area to prevent or reduce fluid contained in venturi reservoir 282 from easily passing through filter drain hole 288 in the direction of filter 287. In certain embodiments, the cross-sectional area of filter drain hole 288 is about 10% or less of the cross-sectional area of vacuum port 283.

Venturi reservoir 282 includes a level sensor area 289 defined in base 206. Vacuum port 283 is disposed above level sensor area 289. Level sensor area 289 is disposed along an optical path of an infrared light sensor in console 100 that is used to determine a fluid level in venturi reservoir 282. In certain embodiments, the infrared light sensor is a single camera sensor or a complementary metal oxide semiconductor (CMOS) sensor. During normal operation, a nominal fluid level in venturi reservoir 282 is between lower and upper limits of level sensor area 289. The opening corresponds to level sensor area 289. The relatively small size or footprint of level sensor area 289 enables the use of a more compact housing 205 compared to other designs.

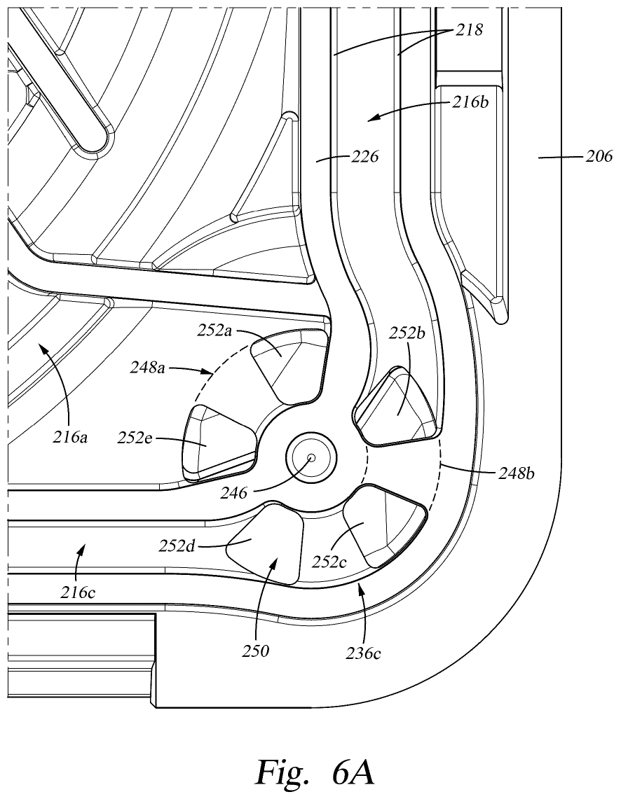

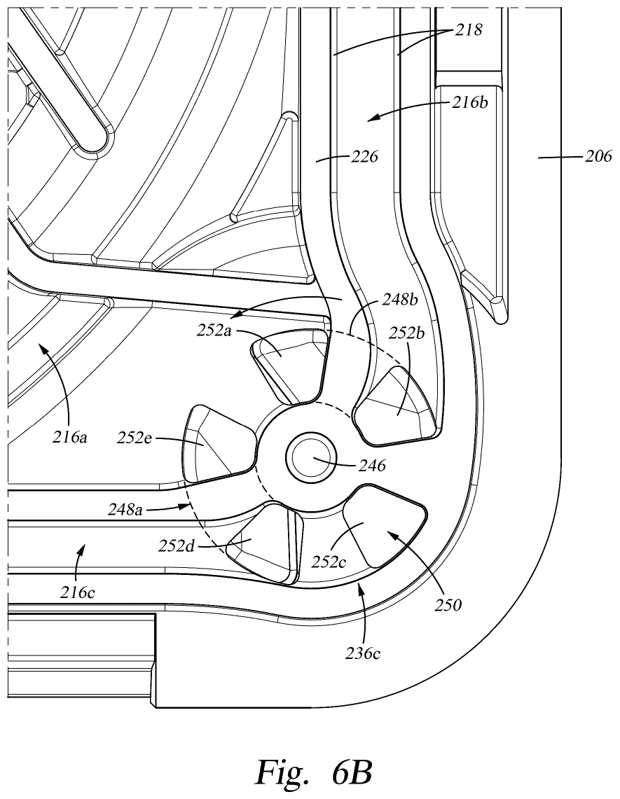

In certain embodiments, venturi reservoir 282 receives fluids that are aspirated through port 210a of base 206. For example, aspirated fluids may enter port 210a, flow through channel 216c to port 252d, and then enter into venturi reservoir 282 through port 252e. Thus, port 252e is in fluid communication with an aspiration line of surgical handpiece 112 (shown in FIG. 1A) through port 210a of base 206. Port 252e is disposed through base 206 below level sensor area 289. Note that ports 252d-e are also referred to as “fourth port” and “fifth port,” respectively, with regard to the description of the valve in conjunction with FIGS. 6A-6B.

In certain embodiments, venturi reservoir 282 receives fluids that are suctioned through port 292 on the front side of handle 208b. For example, fluids may enter port 292, flow through channel 293 in handle 208b to port 294a in base 206, and then enter into venturi reservoir 282 through port 294b. Port 294b is disposed through base 206 above level sensor area 289. In certain embodiments, fluids entering venturi reservoir 282 through port 252e or port 294b include a mixture of liquid, such as BSS (balanced salt solution), and air. In some other embodiments, the fluid is only liquid or only air. In certain embodiments, a flow rate of the fluid is about 200 cc/min (cubic centimeters per minute) or less.

The presence of air bubbles in or near level sensor area 289 interferes with accurate detection of the fluid level in venturi reservoir 282 because the air bubbles obscure the air-liquid interface. Therefore, in certain embodiments, a plurality of air baffles 295 (295a-c) are disposed within venturi reservoir 282 in order to divert air bubbles away from level sensor area 289. The plurality of air baffles 295 are integral with base 206 and contact cover 208a when cover 208a is coupled to base 206. In some other embodiments, the plurality of air baffles 295 are integral with cover 208a instead of base 206.

A lower air baffle 295a is disposed above port 252e and extends up and to the left of the viewer in FIG. 2E. An upper air baffle 295b is disposed below port 294b and extends down and to the left of the viewer in FIG. 2E. Air baffles 295a-b form corresponding channel-like structures within venturi reservoir 282 that begin at corresponding ports 252e, 294b and end on the left side of venturi reservoir 282. For example, first channel 216a corresponds to a portion of venturi reservoir 282 below lower air baffle 295a.

A central air baffle 295c is located to the right of the respective ends of air baffles 295a-b and extends from below to above level sensor area 289. The position of central air baffle 295c prevents air bubbles from crossing level sensor area 289 even after the air bubbles pass above lower air baffle 295a and below upper air baffle 295b, respectively. An opening between lower air baffle 295a and central air baffle 295c provides a path for equalization of fluid on the left and right sides of central air baffle 295c, so that the fluid level in level sensor area 289 corresponds to the actual fluid level in venturi reservoir 282. An opening between upper air baffle 295b and central air baffle 295c provides a path for the flow of air bubbles from the left side of central air baffle 295c to vacuum port 283 without crossing level sensor area 289.

A liquid baffle 296 is disposed within venturi reservoir 282 above level sensor area 289. Liquid baffle 296 is configured to reduce or prevent liquid from entering vacuum port 283 in cover 208a, such as during bubbling, frothing, or overrun of the liquid contained in venturi reservoir 282. Like air baffles 295, liquid baffle 296 is integral with base 206 and contacts cover 208a when cover 208a is coupled to base 206. An upper portion of central air baffle 295c extends directly below liquid baffle 296 so that liquid falling down from liquid air baffle 296 is blocked from entering the right side of central air baffle 295c and subsequently prevented from crossing level sensor area 289.

FIGS. 2F-2G are enlarged exploded front side and back side isometric views, respectively, of a portion of surgical cassette 200 of FIG. 2A illustrating an example valve assembly having two passages in the valve body, according to certain embodiments. FIGS. 3A-3D illustrate various views of the valve body having two passages. Accordingly, FIGS. 2F-2G and 3A-3D are described together herein for clarity.

Valve body 236c of third valve assembly 204c has a first end 240, a second end 242, a substantially cylindrical outer surface 244 connecting first end 240 and second end 242, and a longitudinal axis 246 orthogonal to first end 240. Cylindrical outer surface 244 includes multiple stepped portions having different outer dimensions. In some embodiments, the cylindrical surface includes a base portion 245 separated from a drive interface 258 by a first shoulder 262 and separated from a collar portion 247 by a second shoulder 249, the collar portion 247 having a larger diameter relative to the base portion 245 as best seen in FIG. 3C. In one embodiment, the base portion 245 has a diameter of approximately 13 millimeters (mm) and a longitudinal length of 5.21 mm, while the collar portion has a diameter of approximately 16 mm and a longitudinal length of 3.38 mm. Transitioning from collar portion 247 to a valve elastomer 251 is a third shoulder 259.

Valve body 236c is rotatable about longitudinal axis 246. Two passages 248 (248a-b) are formed in valve body 236c at second end 242. In FIG. 2F, first passage 248a and second passage 248b are about equal in length when measured in a circumferential direction about longitudinal axis 246 (e.g., extending about longitudinal axis 246 in a circumferential direction by about 140° (degrees)-150°. In the illustrated embodiments, each passage 248 is sized to simultaneously open fluid communication with two ports of base 206 as described in more detail below respect to FIGS. 6A-6B. In some other embodiments, each passage 248 may be sized to simultaneously open fluid communication with any suitable number of ports (e.g., two, three, or four ports). In the illustrated embodiments, passages 248 include arc-shaped annular segments extending circumferentially about longitudinal axis 246. In certain embodiments, a cross-section of passages 248 may be circular, round, oval, polygonal, square, any other suitable shape, or combinations thereof.

Terminal ends of each passage 248 are defined through first end 240 of valve body 236c. In certain embodiments, a center axis of each passage 248 at the terminal ends is parallel to longitudinal axis 246. In certain embodiments, at least a portion of each passage 248, e.g., the portion between the terminal ends, is orthogonal to longitudinal axis 246. In certain embodiments, during fabrication, passages 248 are machined or molded in a direction parallel to longitudinal axis 246, e.g., starting from second end 242. In other words, an entire surface of each passage 248 is visible from second end 242 when viewed in a direction parallel to longitudinal axis 246. In the illustrated embodiments, passages 248 include an equal flow area. In some other embodiments, passages 248 may have different flow areas. In certain other embodiments, there may only be one passage as shown in FIGS. 4A-4D, or more than two passages formed in the valve body.

Five ports 252 are formed through base 206 as shown in FIG. 2G. In operation, valve body 236c is rotatable relative to the first side 234c of base 206 to align each passage 248 with a corresponding port 252 (252a-e) of base 206 to open pressure and/or fluid communication between corresponding ones of the plurality of channels 216. A flow axis through each port 252 is parallel to longitudinal axis 246 of valve body 236c. A shape of each port 252 may correspond to a cross-section of each passage 248 of valve body 236c to help maintain laminar flow therethrough. In certain embodiments, a cross-sectional shape of each passage 248 may be formed by continuing a shape of the corresponding port 252 as a swept surface through valve body 236c.

In the illustrated embodiments, five ports 252 are formed through base 206 within each bore 230. However, there may be any suitable number of ports (e.g., two to seven ports) in each bore 230. In the illustrated embodiments, ports 252 include arc-shaped annular segments. In some other embodiments, ports 252 may be circular, round, oval, polygonal, square, any other suitable shape, or combinations thereof. In the illustrated embodiments, ports 252 have an equal flow area. In some other embodiments, ports 252 may have different flow areas. In the illustrated embodiments, ports 252 are uniformly spaced in the circumferential direction. In some other embodiments, ports 252 may have different spacing in the circumferential direction.

Retaining ring 238c has an annular body 254 with a center opening 256. Retaining ring 238c fits over and around valve body 236c such that a drive interface 258 (shown in FIG. 2G) of valve body 236c is received within center opening 256. In some embodiments, drive interface 258 engages a drive mechanism of console 100 for rotating valve body 236 about longitudinal axis 246. Annular body 254 includes multiple stepped portions having different outer dimensions. As an example, the annular body 254 has an outer diameter (e.g., outer shoulder 260a) of approximately 18 mm and an inner diameter (e.g., inner shoulder 260b) of about 14 mm. At least one portion of annular body 254 is disposed radially between outer cylindrical surface 244 of valve body 236c and cylindrical inner wall 232c of bore 230c. At least another portion of annular body 254 is disposed outside bore 230c. An outer shoulder 260a formed between the stepped portions of annular body 254 contacts back side 212 of base 206 when retaining ring 238c is fully seated in bore 230c.

In certain embodiments, valve body 236 includes an orientation-identifying feature, or hard stop feature, which can be used to correlate a rotational state of valve body 236 with one of the base 206 or retaining ring 238 in order to ensure proper alignment between passages 248 and corresponding ports 252 during operation. In FIGS. 2F-2G, the hard stop feature includes a first profile 264 formed on cylindrical outer surface 244 of valve body 236 which is configured to contact a corresponding second profile 266 formed on annular body 254 of retaining ring 238c. Further rotation of valve body 236 is prevented by contact between first profile 264 and corresponding second profile 266. As shown in FIG. 2G, second profile 266 is disposed in a corresponding notch 268 formed on cylindrical inner wall 232c of bore 230c so that rotational alignment of retaining ring 238c is fixed relative to base 206 which is necessary for functioning of the hard stop feature. In some embodiments, the retaining rings 238 disclosed and described above in relation to the valve bodies 236 may also be used in conjunction with a single-passage valve body, for example, as described in further detail with reference to FIGS. 4A-4D.

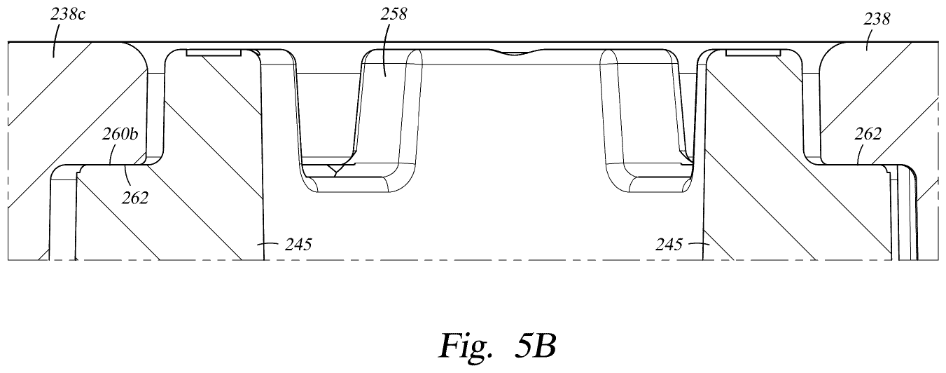

Valve body 236c includes a valve elastomer 251 (best seen in FIG. 3A) at second end 242 which rotatably contacts first side 234c of base 206 (shown in FIGS. 2G and 5A) for sealing second end 242 with first side 234c. Valve elastomer 251 has a first side 270 coupled to second end 242 of valve body 236c, and a second side 272 configured to engage with first side 234c. Sealing between second end 242 and first side 234c of base 206 forms a sealing interface between the planar (e.g., non-cylindrical) surfaces of second end 242 and first side 234c. Because the sealing interface is on a longitudinal end (i.e., second end 242) of valve body 236c, this sealing arrangement may be referred to as end-sealing. In certain embodiments, valve elastomer 251 is formed from a rubber or elastomeric material (e.g., silicone rubber) which is bonded (e.g., over-molded) onto valve body 236c at second end 242. In some other embodiments, valve body 236c and valve elastomer 251 may be integrally formed from the same material (e.g., a high density polyethylene or the like).

Turning to FIG. 3C, in certain embodiments, second end 242 of valve body 236c includes recessed portions 276 (276a-b) configured to engage and/or align with corresponding protruding portions 274 (274a-b) on first side 270 of valve elastomer 251 to couple valve elastomer 251 to valve body 236c. In certain embodiments, recessed portions 276 function as anchors that restrain protruding portions 274 when protruding portions 274 are formed during an over-molding process to fabricate the valve elastomer 251 over valve body 236c, thereby bonding the valve elastomer 251 and valve body 236c. In certain embodiments, the recessed portions 276 include a first, outer recessed portion 276a that may be continuously disposed or formed around a perimeter of second end 242. The recessed portions 276 may further include a second, inner recessed portion 276b that may be continuously disposed or formed around a lateral center point of second end 242 of valve body 236c.

In some embodiments, first recessed portion 276a may be equidistant from second recessed portion 276b around an area between recessed portions 276a and 276b. In FIG. 3C, first recessed portion 276a is configured to engage with a first protruding portion 274a (outer protruding portion) of valve elastomer 251, and second recessed portion 276b is configured to engage with a second protruding portion 274b (inner protruding portion) of valve elastomer 251.

Other quantities and arrangements of recessed portions on second end 242 and/or protruding portions on valve elastomer 251 are also contemplated for anchoring valve elastomer 251 to valve body 236c. For example, second end 242 of valve body 236c may include one or more recessed portions (e.g., 3-4 recessed portions, 4-5 recessed portions, 6-7 recessed portions, or 7-8 recessed portions), such that valve elastomer 251 may have a corresponding number of protruding portions. Additionally, although protruding portions 274 of valve elastomer 251 and corresponding recessed portions 276 of valve body 236c are shown as being rectangular in FIG. 3C, the protruding portions and corresponding recessed portions may be circular, triangular, trapezoidal, any other suitable coupling shape, or combination thereof. In some embodiments, recessed portions 276 can extend from the third shoulder 259 all the way to the second shoulder 249, such that recessed portions 276 may be through holes functioning as anchors.

In some embodiments, some or all of the recessed portions 276 may be discontinuous, such that one or more individual recessed portions may be placed at a location on second end 242 of valve body 236c (e.g., around perimeter of second end 242). For example, second end 242 of valve body 236c may include one recessed portion longitudinally opposite of recessed surface 250 or two or more recessed portions spaced equally around the perimeter of second end 242. In some embodiments, the second end 242 of valve body 236c comprises one or more recessed portions laterally opposite of recessed surface 250.

Second side 272 of valve elastomer 251 includes at least one recessed surface 250 surrounded by protruding sealing portions 280. In some embodiments, recessed surface 250 is configured to decrease ballooning of valve elastomer 251 into ports 252 of base 206, which can cause wear and stress on valve elastomer 251 and other mating components, while also increasing the torque. As such, recessed surface 250 reduces mechanical stress across the valve elastomer throughout shelf life and during rotation of the valve assembly.

In certain embodiments, a lubricant (e.g., silicone oil) may also be used to facilitate relative rotation between interfacing surfaces of valve body 236c (e.g., first shoulder 262) and retaining ring 238c (e.g., inner shoulder 260b) as described in more detail with reference to FIG. 5B. In certain embodiments, lubricant may be dispensed onto one or more interfacing surfaces. As an example, lubricant is used to reduce torque and facilitate rotation of valve assembly 204c within bore 230c. In such an example, the lubricant is also dispensed onto a sealing interface between valve elastomer 251 of valve assembly 204c and the first side 234c of base 206.

In some embodiments, valve elastomer 251 can be impregnated with a lubricant (e.g., silicone oil) which eliminates the need of dispensing a lubricant on sealing surfaces. In other words, valve elastomer 251 is coated with a self-lubricating material (e.g., a slick silicone LSR (Liquid Silicone Rubber) coating) or is a self-lubricating silicone rubber. As such, valve elastomer 251 is configured to decrease a coefficient of friction.

Recessed surface 250 is generally planar and orthogonal to longitudinal axis 246. Recessed surface 250 of valve elastomer 251 is adjacent to a passage (e.g., passage 248a or 248b) through valve elastomer 251 and valve body 236c. In certain embodiments, as shown in FIG. 3D, the recessed surface 250 is bounded by a first semi-circular inner edge 290a, two straight sides 290b from the inner edge 290a to an area of largest width 290c of recessed surface 250, and a second semi-circular outer edge 290d. In some embodiments, recessed surface 250 of valve elastomer 251 can extend about longitudinal axis 246 in either circumferential direction between about 20° and about 90° (e.g., between 35° and 75°). In some embodiments, recessed surface 250 of valve elastomer 251 is surrounded by protruding sealing portions 280 of second side 272 of valve elastomer 251 that extend past recessed surface 250, such that recessed surface 250 may be an interior area of valve elastomer 251. In such an embodiment, recessed surface 250 may not extend to a perimeter of valve elastomer 251.

In some embodiments, recessed surface 250 may be defined as an area of an outer surface that is recessed from protruding sealing portions 280 on second side 272 of valve elastomer 251 by a maximum height of, for example, 0.152 mm, and that has a cross sectional width of, for example, 4.80 mm. In some embodiments, the passages 248 may be substantially identical and have cross sectional widths of, for example, 2.67 mm. In some embodiments, the valve elastomer 251 has an initial height of, for example, 1.52 mm which in certain embodiments may be compressible when a force is applied.

Although valve elastomer 251 is shown as including a recessed surface 250 on second side 272, in some embodiments, second side 272 of valve elastomer 251 has a generally planar morphology, without any recessed surfaces. While recessed surface 250 of valve elastomer 251 is shown as being a flat, linear surface in FIG. 3C, recessed surface 250 may also be curved, angled relative to longitudinal axis 246, or any combination thereof. Additionally, there may be more than one recessed surface (e.g., 2-3 recessed surfaces, 4-5 recessed surfaces, or 5-6 recessed surfaces). By incorporating recessed surface 250 in valve elastomer 251, compression at certain areas of valve assembly 204c may be decreased which further reduces reaction force within valve assembly 204c.

Returning now to FIGS. 2A-2D, in some embodiments, valve assemblies 204a-204d may comprise a combination of both multiple-passage valve bodies 236 and single-passage valve bodies 402. For example, first valve assembly 204a and third valve assembly 204c as seen in FIG. 2B may each comprise a multiple-passage valve body 236 which are each in pressure and/or fluid communication with first pump assembly 202a and port 210a to provide aspiration (suction) through port 210a and ventilation during an operation respectively, while the second valve assembly 204b and fourth valve assembly 204d each comprise a single-passage valve body (shown in FIGS. 4A-4D) which are each in pressure and/or fluid communication with second pump assembly 202b and port 210c to provide irrigation (infusion) through port 210c and bypass during the same operation, respectively.

FIGS. 4A-4D illustrate an exemplary embodiment of valve assembly 404, which is an alternative embodiment of valve assembly 204. Valve assembly 404 comprises a valve body 436 and a valve elastomer 451 with one passage 448. Valve body 402 has a first end 440, a second end 442, a cylindrical outer surface 444 connecting first end 440 and second end 442, and a longitudinal axis 446 orthogonal to first end 440. Single passage 448 through valve elastomer 451 and valve body 436 is formed at second end 442.

In an embodiment shown in FIG. 4A, passage 448 extends about longitudinal axis 446 in a circumferential direction by about 210°. In the illustrated embodiments, passage 448 is sized to simultaneously open fluid communication with three ports of base 206. In some other embodiments, passage 448 may be sized to simultaneously open fluid communication with any suitable number of ports (e.g., two, three, or four ports).

In FIG. 4B, valve body 436 further comprises a drive interface 458 which engages a drive mechanism of console 100 for rotating valve body 436 about longitudinal axis 446. In certain embodiments, first end 440 comprises a first shoulder 462, and second end 442 comprises a second shoulder 449 and a third shoulder 459. Cylindrical outer surface 444 includes multiple stepped portions having different outer dimensions. In some embodiments, cylindrical outer surface 444 is comprised of a base portion 445 separated from drive interface 458 by first shoulder 462 and separated from a collar portion 447 by a second shoulder 449, collar portion 447 having a larger diameter relative to base portion 445. In one embodiment, the base portion 445 has a diameter of approximately 13 mm and a longitudinal length of 5.21 mm, while the collar portion has a diameter of approximately 16 mm and a longitudinal length of 3.38 mm. Transitioning from collar portion 447 to valve elastomer 451 is third shoulder 459.

In the illustrated embodiments, passage 448 includes an arc-shaped annular segment extending circumferentially about longitudinal axis 446. In certain embodiments, a cross-section of passage 448 may be circular, round, oval, polygonal, square, any other suitable shape, or combinations thereof. Terminal ends of the passage 448 are defined through second end 442 of valve body 436. In certain embodiments, a center axis of the passage 448 at the terminal ends is parallel to longitudinal axis 446. In certain embodiments, at least a portion of the passage 448, e.g., the portion between the terminal ends, is orthogonal to longitudinal axis 446. In certain embodiments, during fabrication, passage 448 is machined or molded in a direction parallel to longitudinal axis 446, e.g., starting from second end 442. In other words, an entire surface of the passage 448 is visible from second end 442 when viewed in a direction parallel to longitudinal axis 446. In the illustrated embodiments, passage 448 includes an equal flow area. In some other embodiments, passage 448 may have different flow areas.

As mentioned above, valve assembly 404 includes valve elastomer 451 at second end 442 which rotatably contacts first side 234c of base 206 for sealing second end 442 with first side 234c, similar to valve body 236c shown in FIGS. 2G, 3A-3C, and 5A. Similar to valve elastomer 251 shown in FIGS. 3A-3C, valve elastomer 451 comprises a first side 470 coupled to second end 442 of valve body 436, and a second side 472 configured to engage with first side 234c. Sealing between second end 442 and first side 234c of base 206 forms a sealing interface between the planar (e.g., non-cylindrical) surfaces if second end 242 and first side 234c. Because the sealing interface is on a longitudinal end (i.e., second end 442) of valve body 436, this sealing arrangement may be referred to as end-sealing. In certain embodiments, valve elastomer 451 is formed from a rubber or elastomeric material (e.g., silicone rubber) which is bonded (e.g., over-molded) on valve body 436 at second end 442. In some other embodiments, valve body 436 and valve elastomer 451 may be integrally formed from the same material (e.g., high density polyethylene).

Turning to FIG. 4C, in certain embodiments, second end 442 of valve body 436c includes recessed portions 476 (476a-c) which are configured to engage and/or align with corresponding protruding portions 474 (474a-c) of first side 470 of valve elastomer 451 to couple valve elastomer 451 to valve body 436. In certain embodiments, recessed portions 476 function as anchors that restrain protruding portions 474 when protruding portions 474 are formed during an over-molding process to fabricate the valve elastomer 451 over valve body 436, thereby bonding the valve elastomer 451 and valve body 436.

In certain embodiments, the recessed portions 276 include a first, outer recessed portion 476a that may be continuously disposed or formed around a perimeter of second end 442. The recessed portions 476 may further include a second, inner recessed portion 476b that may be continuously disposed or formed around a lateral center point of second end 442 of valve body 436. The recessed portions 476 may further include a third, middle recessed portion 476c that may be disposed or formed between first recessed portion 476a and second recessed portion 476b, and that corresponds to recessed surface 450. In some embodiments, first recessed portion 476a may be equidistant from second recessed portion 476b around an area between recessed portions 476a and 476b.

In FIG. 4C, first recessed portion 476a is configured to engage with a first protruding portion 474a (outer protruding portion) of valve body 436, second recessed portion 476b is configured to engage with a second protruding portion 474b (inner protruding portion) of valve body 436, and third recessed portion 476c is configured to engage with a third protruding portion 474c (middle protruding portion) of valve body 436.

Other quantities and arrangements of recessed portions on second end 442 and/or protruding portions on valve elastomer 451 are also contemplated for anchoring valve elastomer 451 to valve body 236. For example, second end 442 of valve body 436 may include one or more recessed portions (e.g., 4-5 recessed portions, 5-6 recessed portions, 6-7 recessed portions, or 7-8 recessed portions), such that valve elastomer 451 may have a corresponding number of protruding portions. Additionally, although protruding portions 474 of valve elastomer 451 and corresponding recessed portions 476 of valve body 436 are shown as being rectangular, the protruding portions and corresponding recessed portions may be circular, triangular, trapezoidal, any other suitable coupling shape, or combination thereof. In some embodiments, recessed portions 476 can extend from the third shoulder 459 all the way to the second shoulder 449, such that recessed portions 476 may be through holes functioning as anchors.

In some embodiments, some or all of recessed portions 476 may be discontinuous, such that one or more individual recessed portions may be placed at a location on second end 442 of valve body 436 (e.g., around the perimeter of second end 442). For example, second end 442 of valve body 436 may include one recessed portion laterally opposite of recessed surface 450 or two or more recessed portions spaced equally around the perimeter second end 442. In some embodiments, the second end 442 of valve body 436 comprises one or more recessed portions longitudinally opposite of recessed surface 450.

Second side 472 of valve elastomer 451 includes a recessed surface 450 surrounded by protruding sealing portions 480. In some embodiments, recessed surface 450 is configured to decrease ballooning of valve elastomer 451 into ports 252 of base 206, which can cause wear and stress on valve elastomer 451 and other mating components, while also increasing the torque. Recessed surface 450 of valve elastomer 451 is orthogonal to longitudinal axis 446. Recessed surface 450 of valve elastomer 451 is adjacent to passage 448 through valve elastomer 451 and valve body 436.

In certain embodiments, as shown in FIG. 4D, the recessed surface 450 is bounded by a first semi-circular inner edge 490a, two straight sides 490b from the first semi-circular inner edge 490a to an area of largest width 490c of the recessed surface 450, and a second semi-circular outer edge 490d. In some embodiments, recessed surface 450 of valve elastomer 451 extends about longitudinal axis 446 in either circumferential direction between about 20° and about 180° (e.g., between 55° and 145°. In some embodiments, recessed surface 450 of valve elastomer 451 is surrounded by protruding sealing portions 480 of second side 472 of valve elastomer 451 that extend past recessed surface 450, such that recessed surface 450 may be an interior area of valve elastomer 451. In such an embodiment, a gap exists between recessed surface 450 and an outer perimeter of valve elastomer 451.

In some embodiments, recessed surface 450 may be defined as an area of an outer surface that is recessed from protruding sealing portions 480 on second side 472 of valve elastomer 451 by a maximum height of, for example, 0.152 mm, and that has a cross sectional width of, for example, 4.80 mm. In some embodiments, passage 448 has a cross sectional width of, for example, 2.67 mm. In some embodiments, the valve elastomer 451 has an initial height of 1.52 mm which in certain embodiments may be compressible when a force is applied.

Although valve elastomer 451 is shown as including a recessed surface 450 on second side 472, in some embodiments, second side 472 of valve elastomer 451 has a generally planar morphology, without any recessed surfaces. While recessed surface 450 of valve elastomer 451 is shown as being a flat, linear surface in FIG. 4C, recessed surface 450 may also be curved, angled relative to longitudinal axis 446, or any combination thereof. Additionally, there may be more than one recessed surface (e.g., 2-3 recessed surfaces, 4-5 recessed surfaces, or 5-6 recessed surfaces). By incorporating recessed surface 450 in valve elastomer 451, compression at certain areas of valve assembly 404c may be decreased which further reduces reaction force within valve assembly 404c. Greater detail of the interaction between a valve elastomer and first side of base is described with reference to FIG. 5A. Although FIG. 5A is described herein with reference to valve assembly 204 shown in FIGS. 2A-2G and 3A-3D, valve assembly 404 shown in FIGS. 4A-4D may be implemented similarly. When viewed in cross-section, valve elastomer 251 forms a fluidic seal (e.g., fluidic sealing interface) with first side 234 of base 206 of housing 205 as detailed.

Turning now to FIG. 5B, a top portion of the valve assembly 204 is illustrated. As shown, an inner shoulder 260b formed between the stepped portions of annular body 254 contacts first shoulder 262 of valve body 236c radially surrounding drive interface 258 to apply a sealing force on valve body 236c. In certain embodiments, a lubricant (e.g., silicone oil) may be used to facilitate relative rotation between interfacing surfaces of valve body 236c (e.g., first shoulder 262) and retaining ring 238c (e.g., inner shoulder 260b). In certain embodiments, lubricant may be dispensed onto one or more interfacing surfaces. In some embodiments, the lubricant may be any copolymer liquid comprising dimethylisiloxane and trifluoropropylmethylsiloxane that provides a lubricious coating.

The sealing force applied by retaining ring 238c is applied in a direction parallel to longitudinal axis 246 (axially) and forces second end 242 of valve body 236c towards first side 234c of base 206 which compresses valve elastomer 251 against first side 234c of base 206 thereby forming a fluidic seal between passages 248 and corresponding ports 252. In certain embodiments, valve elastomer 251 is compressed in the direction parallel to longitudinal axis 246 (axially) up to 34% of its total height, thereby compressing or reducing the overall height down to approximately 1 mm, when retaining ring 238c is fully seated in bore 230c.

In certain embodiments, retaining ring 238c is coupled to base 206 using a solid-state welding technique (e.g., ultrasonic welding) so that no gap or space is present between the inner shoulder 260b of the retaining ring 238c and the first shoulder 262 of the valve body 236. In some other embodiments, retaining ring 238c may be snap-fit, threaded, and/or adhered to base 206. When valve elastomer 251 is compressed by retaining ring 238c, the recessed surface 250 may also mitigate and/or reduce ballooning of valve elastomer 251 into ports 252 of housing 205.

FIGS. 6A-6B are front side elevation views of a portion of surgical cassette 200 of FIG. 2A illustrating two different valve positions, according to certain embodiments. In FIGS. 6A-6B, cover assembly 208 is omitted for clarity. A first valve rotational state of valve assembly 204c is illustrated in FIG. 6A. In the first valve rotational state, first passage 248a (indicated with dashed outline) of valve body 236c is aligned with first port 252a and fifth port 252e of venturi reservoir 282. When first passage 248a is aligned with both ports of venturi reservoir 282, fluid communication with venturi reservoir 282 is closed, and therefore, the first port 252a and fifth port 252e may be referred to as “closed” in the first valve rotational state.

First port 252a is adjacent to fifth port 252e and is disposed through base 206 below level sensor area 289 like fifth port 252e as described above. In the first valve rotational state, second passage 248b (indicated with dashed outline) is aligned with a second port 252b corresponding to a second channel 216b and a third port 252c corresponding to a third channel 216c, thereby opening fluid communication between second channel 216b and third channel 216c. In the first valve rotational state, a flat segment of valve elastomer 251 in a clockwise direction relative to second passage 248b is aligned with a fourth port 252d, thereby closing fluid communication through fourth port 252d.

In the first valve rotational state, a hard prime may be performed on second channel 216b upstream of first pump assembly 202a (also referred to as an “aspiration path” of surgical cassette 200). In general, the hard prime involves building-up vacuum very high in the aspiration path and then suddenly opening the aspiration path to liquid from venturi reservoir 282. The sudden rush of liquid causes a high flow rate through the aspiration path and shears any trapped air bubbles from their positions. The trapped air bubbles are allowed to enter the main flow stream and are passed to a drain bag coupled to surgical cassette 200. In certain embodiments, the hard prime is an automated sequence performed by computer 103 of console 100. The hard prime may be performed as part of the surgical setup of surgical cassette 200. The hard prime provides lower and more repeatable total fluid compliance (improved vacuum responsiveness) of surgical cassette 200 and results in lower fluid volume surges during post-occlusion break surge events.

In certain embodiments, second channel 216b is in fluid communication with first pump assembly 202a (also referred to as an “aspiration pump”) and third channel 216c is in fluid communication with an aspiration line to a surgical site for aspirating fluid from the eye (e.g., via handpiece 112 shown in FIG. 1A). In such embodiments, the first rotational state corresponds to an aspiration state.

An example operation of valve assembly 204c is described below. In certain embodiments, occlusion or blockage of fluid flow through the handpiece can occur during aspiration due to build-up of surgical material (e.g., lens fragments) causing vacuum pressure to build-up between the handpiece and first pump assembly 202a. With sufficient vacuum pressure build-up, breakage of the occlusion often occurs leading to a rapid surge of fluid being aspirated from the eye and into valve assembly 204c through third channel 216c, and in certain embodiments, including second channel 216b and first pump assembly 202a. This process is commonly referred to as post-occlusion break surge.

The occurrence of post-occlusion break surge may be mitigated using certain embodiments disclosed herein. In certain embodiments, post-occlusion break surge is indicated by a rapid pressure change which may be detected using a pressure sensor in the handpiece or the surgical cassette. When post-occlusion break surge is detected, valve assembly 204c may be switched from the first valve rotational state of FIG. 6A to a second valve rotational state illustrated in FIG. 6B by rotating valve body 236c counterclockwise about longitudinal axis 246 by about 72 degrees (as indicated by the arrow).

In the second valve rotational state, first passage 248a (indicated with dashed outline) of valve body 236c is aligned with fourth port 252d and fifth port 252e, thereby opening fluid communication between venturi reservoir 282 and third channel 216c. In the second valve rotational state, second passage 248b (indicated with dashed outline) is aligned with first port 252a and second port 252b, thereby opening fluid communication between venturi reservoir 282 and second channel 216b. In the second valve rotational state, the flat segment of valve elastomer 251 in a clockwise direction relative to second passage 248b is aligned with third port 252c, thereby closing fluid communication through third port 252c.

In embodiments with second channel 216b in fluid communication with first pump assembly 202a and third channel 216c in fluid communication with the surgical site (as described above), the second rotational state corresponds to a dual path venting state. Thus, fluid communication is open between first channel 216a of venturi reservoir 282 and first pump assembly 202a through second channel 216b. In the dual path venting state, fluid communication is also open between first channel 216a of venturi reservoir 282 and the surgical site through third channel 216c. Valve assembly 204c may be switched from the aspiration state shown in FIG. 6A to the dual path venting state shown in FIG. 6B by rotating valve body 236c counterclockwise about longitudinal axis 246 by about 72 degrees (as indicated by the arrow). Switching to the dual path venting state immediately when post-occlusion break surge is detected mitigates the resulting rapid aspiration of fluid from the eye by filling second channel 216b and third channel 216c with fluid before significant fluid flow from the eye occurs.

In the dual path venting state, first port 252a and fifth port 252e are opened simultaneously. Having first port 252a and fifth port 252e opened at the same time provides independent venting to venturi reservoir 282 of vacuum pressure built-up within corresponding channels 216b-c of base 206. For instance, vacuum pressure built-up in third channel 216c between fifth port 252e and the aspiration line is vented through fifth port 252e. Likewise, vacuum pressure built-up in second channel 216b between first port 252a and first pump assembly 202a is vented through first port 252a independently of the venting of the surgical site through the third channel 216c.

In certain embodiments, second channel 216b has a volume of about 4 cc (cubic centimeters), whereas the fluid connection to the surgical site including third channel 216c has a volume of only about 0.5 cc. Thus, the relatively high compliance of second channel 216b contributes a majority of the vacuum pressure volume that leads to the post-occlusion break surge. Therefore, the independent venting that is provided by closing fluid communication between second channel 216b leading to the pump and third channel 216c leading to the eye greatly reduces the post-occlusion break surge volume.

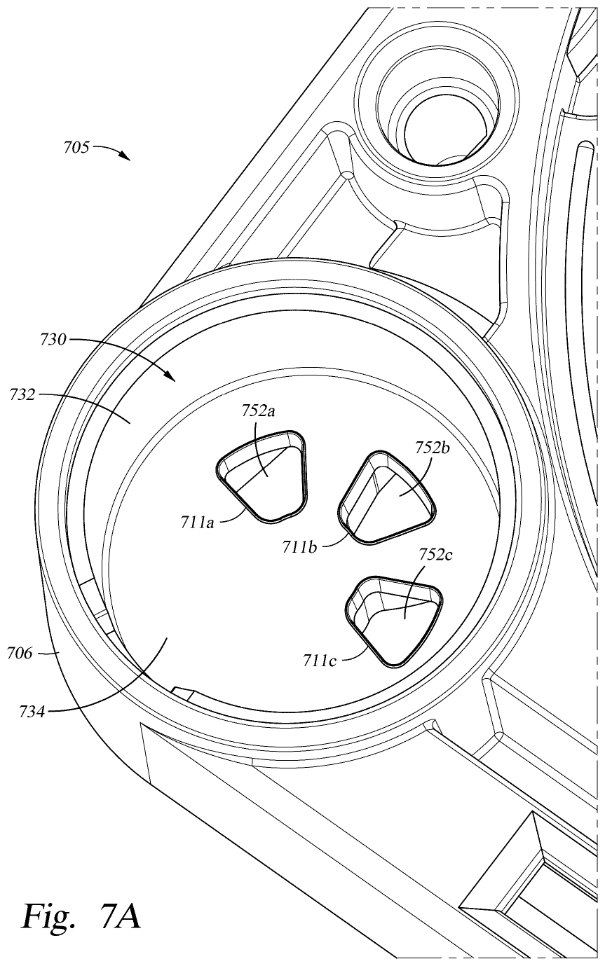

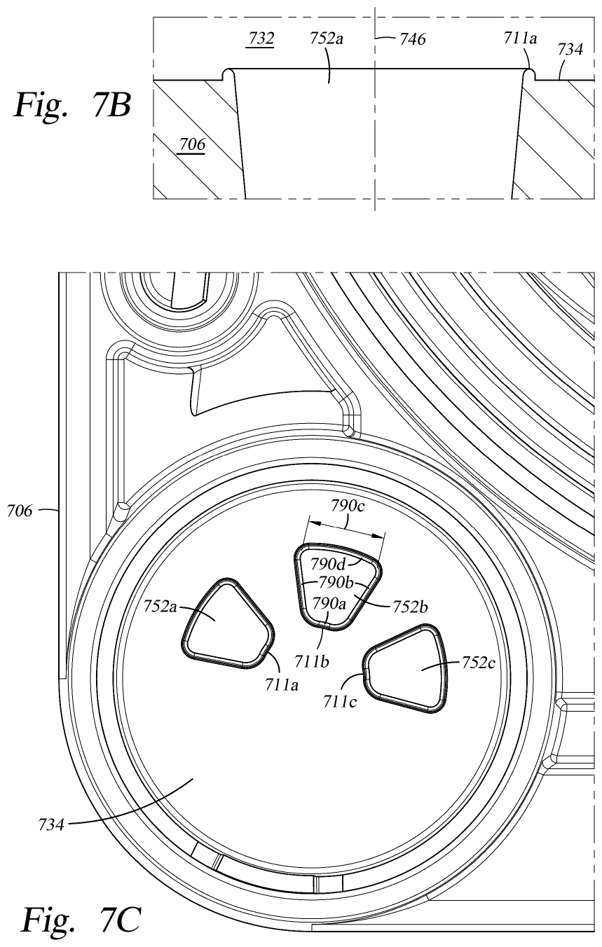

FIGS. 7A-7C illustrate an exemplary embodiment of a housing 705 for receiving a valve assembly. For example, housing 705 may be configured to receive valve assembly 204 (FIGS. 3A-3C), valve assembly 404 (FIGS. 4A-4C), or other valve assembly, e.g., with or without a recessed valve elastomer. Housing 705 may be an alternative embodiment of housing 205 (shown in FIGS. 2A-2G). FIGS. 7A-7C are described together herein for clarity.

Turning to FIG. 7A, an enlarged isometric view of a portion of housing 705 is shown. Similar to housing 205, housing 705 includes a bore 730 formed in base 706 for receiving a corresponding valve assembly. Bore 730 is defined by a cylindrical inner wall 732 and a first side 734. In an embodiment, first side 734 of base 706 is configured to contact a valve assembly. One or more ports (e.g., three shown in FIG. 7A) are formed through base 706. Each port 752 (752a-c) is surrounded by a ring 711 (711a-c) protruding from first side 734 of base 706. Protruding rings 711 are configured to facilitate improved sealing between valve assembly and first side 734 of base 706 of housing 705. For example, protruding rings 711 (may also be referred to as “sealing beads”) are configured to form the seal by engaging with an elastomer (e.g., valve elastomer 251 or 451) of the valve assembly to compress the elastomer against first side 734 of base 706. The ports 752 and protruding rings 711 are described in more detail below with respect to FIGS. 7B-7C.

FIG. 7B illustrates a horizontal cross-section of port 752a, protruding ring 711a, and base 706. As shown in FIG. 7B, protruding ring 711a protrudes from first side 734 of base 706 around perimeter of port 752a. Protruding ring 711a comprises a rounded outer surface that forms a transition between port 752a and first side 734 of base 706. In some embodiments, the rounded outer surface of protruding ring 711a may be, e.g., circular, semi-circular, oval-like, etc. In an embodiment, the cross-section of protruding ring 711a is along an axis 746 of bore 730. In some embodiments, protruding ring 711a has a cross sectional width of, for example, 0.127 mm and extends vertically upwards from first side 734 of base 706 by a maximum height of, for example, 0.254 mm. In some embodiments, protruding rings 711b and/or 711c may have substantially identical dimensions to protruding ring 711a. Although protruding ring 711a is shown as having a rounded cross-section, protruding ring 711a may be rectangular, triangular, trapezoidal, or any other suitable sealing surface.

FIG. 7C illustrates a top perspective view of ports 752, protruding rings 711, and first side 734 of base 706. As best shown in FIG. 7C, protruding rings 711 comprise a first semi-circular inner edge 790a, two straight sides 790b from the inner edge 790a to an area of largest width 790c of the ring, and a second semi-circular outer edge 790d. However, protruding rings 711 are not limited to the embodiment shown in FIGS. 7A-7C, and may be shaped to follow or correspond to a perimeter of ports 752. Other layouts and/or arrangement are also contemplated. For example, protruding rings 711 may be circular, rectangular, triangular, trapezoidal, or any other suitable shape that corresponds to the shape of ports 752. In some embodiments, protruding rings 711 may be a combination of different shapes surrounding perimeters of ports 752.

By incorporating protruding rings 711 on base 706, a valve assembly, e.g., with or without a recessed valve elastomer, can be compressed by retaining ring 238c to a decreased compression because protruding rings 711 provide added compression where it may be needed, and help provide localized sealing to an area around ports 752. Protruding rings 711 may also be used in conjunction with a valve assembly that includes a recessed valve elastomer (e.g., valve assembly 204 or 404) which may be coupled at a same distance from first side 734. As such, protruding rings 711 may afford flexibility in shear weld distance, weld strength, and/or flash control by reducing reaction force. When used with a valve assembly including a recessed valve elastomer, protruding rings 711 may “squeegee” (i.e., help maintain the fluidic seal) the recessed valve elastomer during rotation, which prevents liquid from travelling from one port to another.

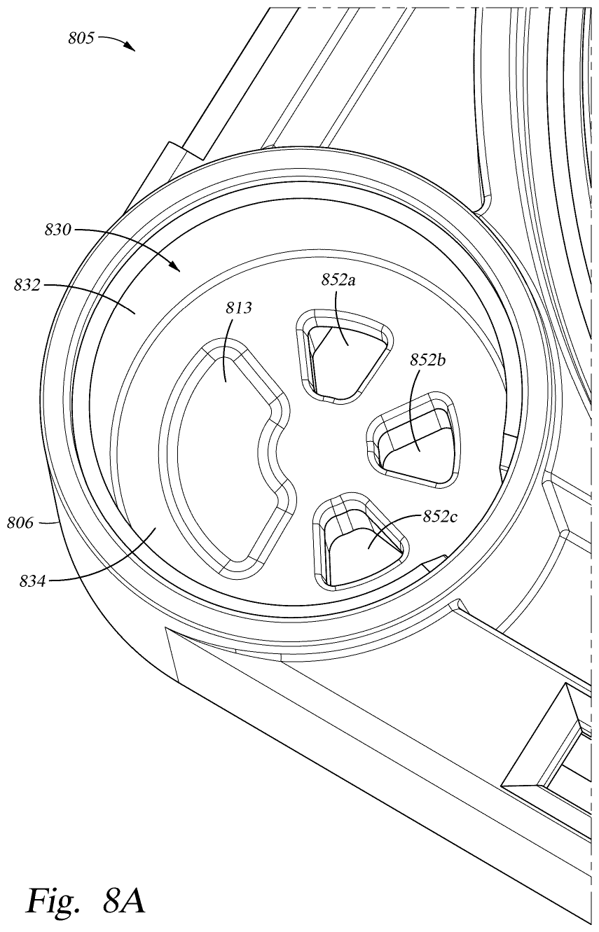

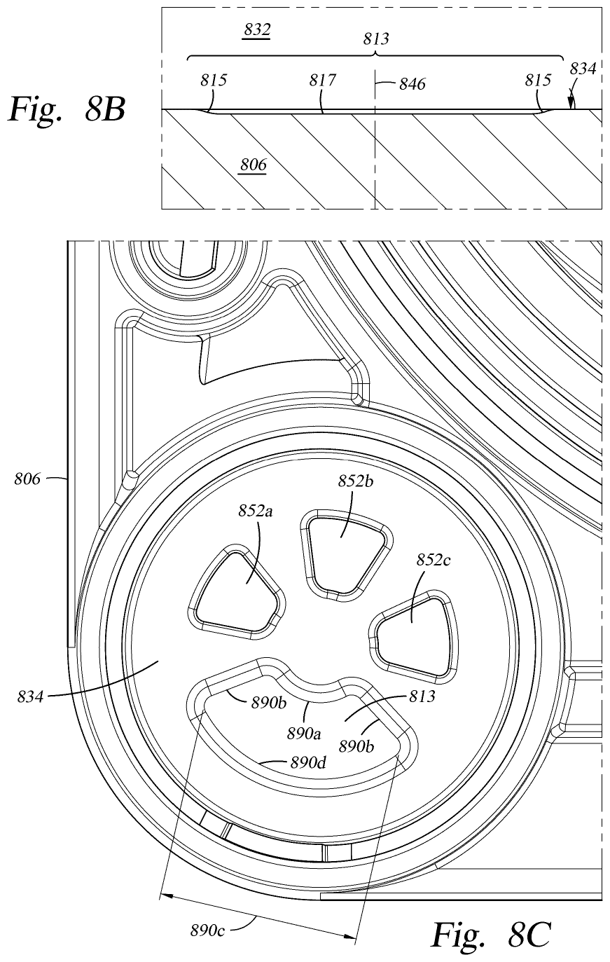

FIGS. 8A-8C illustrate an exemplary embodiment of a housing 805 for receiving a valve assembly. For example, housing 805 may be configured to receive valve assembly 204 (FIGS. 3A-3C), valve assembly 404 (FIGS. 4A-4C), or a standard valve assembly, e.g., that does not include a recessed valve elastomer. Housing 805 may be an alternative embodiment of housing 205 (FIGS. 2A-2G). FIGS. 8A-8C are described together herein for clarity.

FIG. 8A is an enlarged isometric view of a portion of an alternative embodiment of base 706 and housing 705 of FIGS. 7A-7C. Similar to housing 705, housing 805 shown in FIG. 8A comprises a bore 830 formed in base 806 for receiving a corresponding valve assembly. Bore 830 is defined by a cylindrical inner wall 832 and a first side 834. One or more ports (e.g., three shown in FIG. 8A) are formed through base 806. Generally, first side 834 of base 806 is configured to contact a valve assembly. In the embodiments shown, base 806 comprises a recessed surface 813 formed in first side 834 which allows for lower reaction force over the shelf-life of the parts. Recessed surface 813 is described in more detail below with respect to FIGS. 8B-8C.

FIG. 8B illustrates a horizontal cross-section of recessed surface 813 of first side 834 of base 806. As shown in FIG. 8B, recessed surface 813 includes a first surface 815 and a second surface 817. First surface 815 is an outer surface that surrounds second surface 817, forms a transition between second surface 817 and the remainder of the first side 834 of base 806, and forms a perimeter of recessed surface 813. In some embodiments, first surface 815 is angled or sloped relative to a longitudinal axis 846. First surface 815 may also be parallel with longitudinal axis 846. Although first surface 815 is shown as an angled and planar surface, first surface 815 may also be curved, rectangular (i.e., disposed at 90° relative to first side 834), or any other suitable shape.

Second surface 817 is an inner surface surrounded by first surface 815. Second surface 817 is recessed from first side 834 of base 806 and is orthogonal to longitudinal axis 846. In some embodiments, second surface 817 is parallel with a lateral axis of first side 834. Although second surface 817 is shown as being a planar surface parallel with first side 834, second surface 817 may be angled, include a curvature, or have any other suitable shape. In an exemplary embodiment, recessed surface 813 has a cross-sectional width of 3.25 mm and recedes from first side 834 of base 806 by a maximum depth of 0.152 mm.

FIG. 8C illustrates a top perspective view of recessed surface 813, ports 852, and first side 834 of base 806. As best shown in FIG. 8C, recessed surface 813 comprises a first semi-circular inner edge 890a formed by first surface 815, two straight sides 890b from the inner edge 890a to an area of largest width 890c of recessed surface 813, and a second semi-circular outer edge 890d formed by first surface 815. In some embodiments, recessed surface 813 is isolated to an area of first side 834 where there are no ports. However, recessed surface 813 is not limited to the embodiment shown in FIGS. 8A-8C and may be shaped according to the number of ports. Additionally, recessed surface 813 may be circular, rectangular, triangular, trapezoidal, or any other suitable shape. In some embodiments, there may be more than one recessed surface (e.g., 1-2 recessed surfaces, 2-3 recessed surfaces, 3-4 recessed surfaces, 4-5, recessed surfaces, of 5-6 recessed surfaces).

By incorporating recessed surface 813 first side 834 of base 806, compression at certain areas of a corresponding valve assembly may be decreased at areas where sealing may not be needed, which further reduces reaction force within the valve assembly. When left in an “open” position for Ethylene Oxide (ETO) sterilization, a large portion of the valve elastomer will be disposed above recessed surface 813, resulting in less stress in the valve elastomer, ultimately prolonging the seal between the valve elastomer and base 806.

Before being used and while still in the packaging, a valve elastomer is pressed up against first side 834 of base 806 by a retaining ring. As such, the retaining ring and/or base may be susceptible to stress relaxation and/or plastic creep (i.e., deflection of components applying compression over time). Additionally, portions above ports 852 may have some regress and/or capability to decompress, but portions above the flat part do not and are susceptible to high compression and reaction force for a duration of shelf-life until use. Thus, recessed surface 813 allows the valve elastomer to not be as compressed in the previously flat areas, thereby providing some relief and/or alleviation to the valve elastomer.

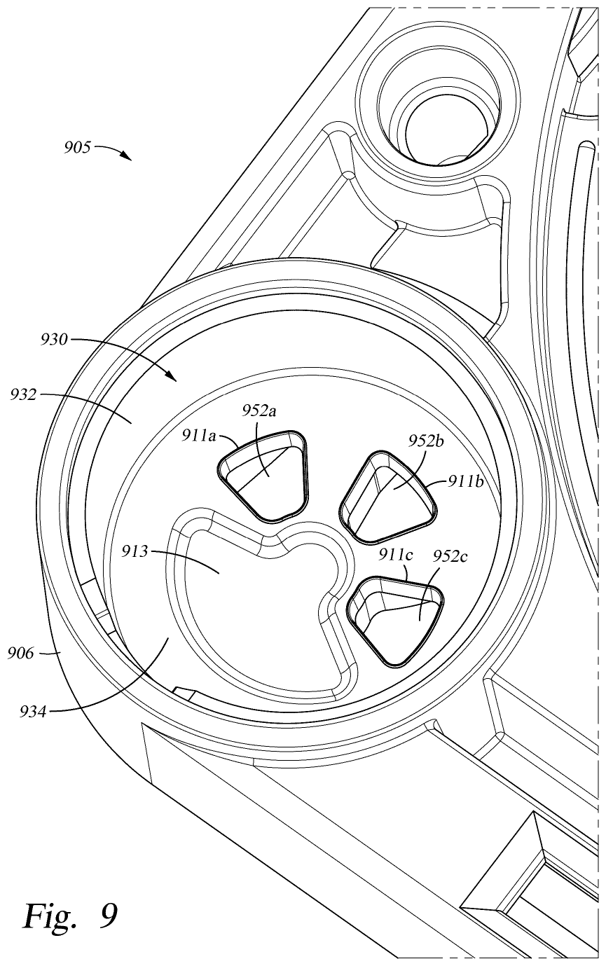

FIG. 9 illustrates an exemplary embodiment of a housing 905 for receiving a valve assembly. For example, housing 905 may be configured to receive valve assembly 204 (FIGS. 3A-3C), valve assembly 404 (FIGS. 4A-4C), or a standard valve assembly, e.g., that does not include a recessed valve elastomer. Housing 905 may be an alternative embodiment of housing 205 (FIGS. 2A-2G) that includes both protruding rings 911 (911a-c) and a recessed surface 913 as described with reference to FIGS. 7A-7C and 8A-8C.

Similar to housing 705 and 805, housing 905 shown in FIG. 9 comprises a bore 930 formed in base 906 for receiving a corresponding valve assembly. Bore 930 is defined by a cylindrical inner wall 932 and a first side 934. One or more ports (e.g., three shown in FIG. 9) are formed through base 906. Each port 952 (952a-c) is surrounded by a ring 911 (911a-c) protruding from first side 934 of base 906. Protruding rings 911 are configured to form a seal between valve assembly and first side 934 of base 906 of housing 905. For example, protruding rings 911 are configured to form the seal by engaging with valve assembly to compress an elastomeric sealing material (e.g., valve elastomer 251 or 451) against first side 934 of base 906.

Base 906 also includes a recessed surface 913 formed in first side 934. Unlike recessed surface 813, recessed surface 913 of FIG. 9 extends into and around a center point of base 906.

Overall, by implementing recessed valve elastomer 251 or 451, protruding rings 711, recessed surface 813, and/or a combination thereof as shown in housing 905, valve assemblies may experience a lower reaction force and/or lower valve torque because such sealing mechanisms may provide adequate compression with less compressive force. As a result, instances of valve abrasion over ports may be reduced, and console motors that provide less torque output may be implemented. Additionally, such sealing mechanisms maintain high valve compression with less ultrasonic weld joint strength, resulting in less melt volume and/or flash. Because less force and/or energy is needed to ultrasonically weld the valve assembly, the surgical cassette may experience less stress. Further, the sealing mechanism described above may reduce chances of valve elastomer abrasion and/or plastic-on-plastic bearing surface abrasion.

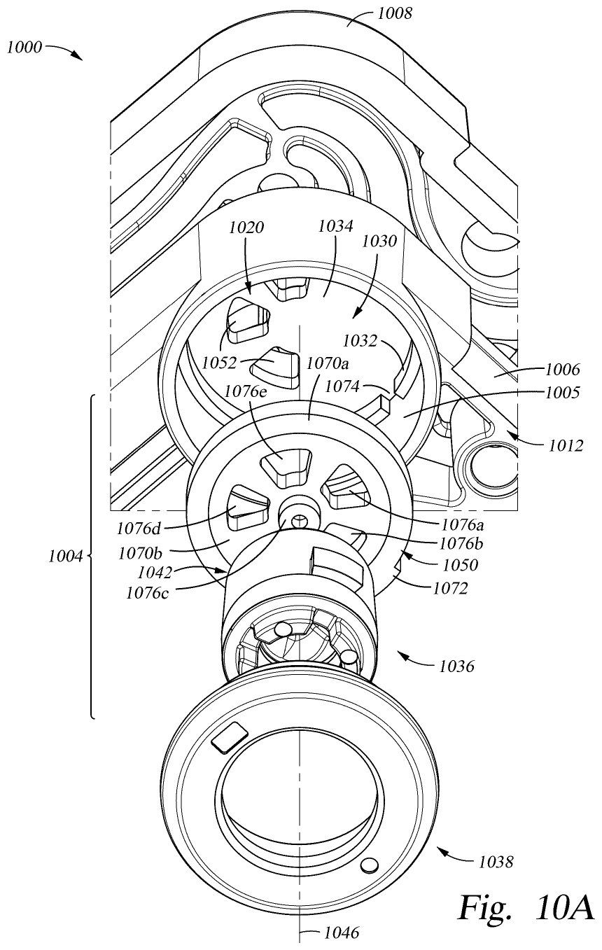

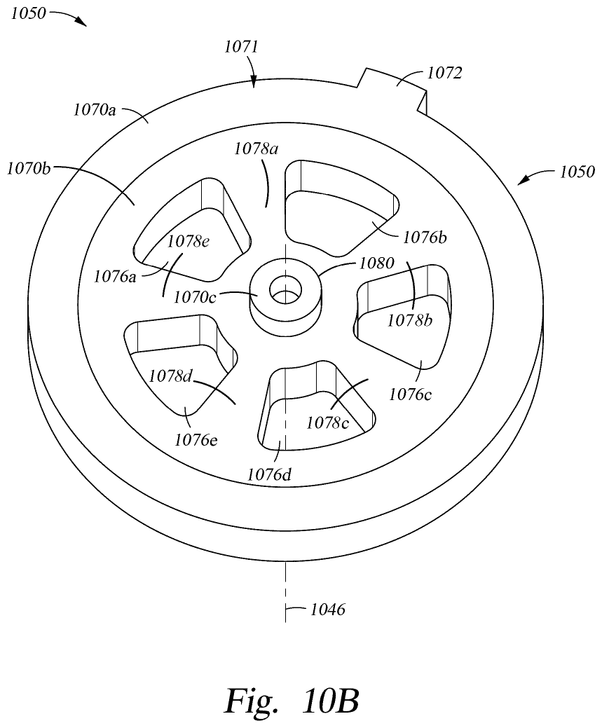

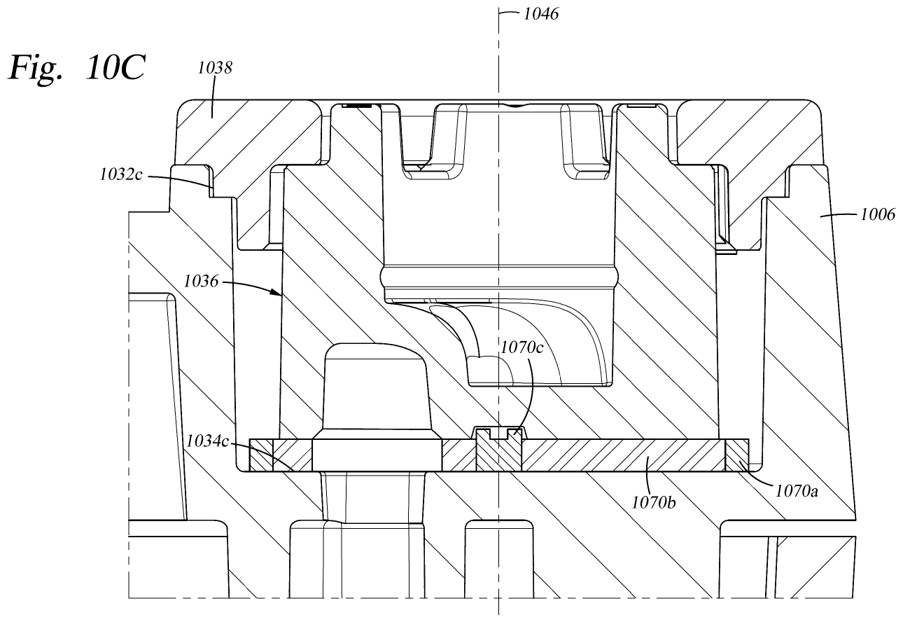

FIGS. 10A-10C illustrate various views of another alternative embodiment of a valve assembly, according to certain embodiments. Turning to FIG. 10A, an exploded back side isometric view of a valve assembly 1004 of a surgical cassette 1000 is shown. Surgical cassette 1000 and valve assembly 1004 are generally similar to surgical cassette 200 and valve assembly 204c, unless otherwise noted below. Surgical cassette 1000 generally includes a base 1006, a cover assembly 1008 coupled to base 1006, and valve assembly 1004 located on back side 1012 of base 1006. A retaining ring 1038 couples a valve assembly 1004 and a valve gasket 1050 to housing 1005. Valve gasket 1050 is described in further detail herein.

Valve gasket 1050 is separate from valve body 1036 and is disposed between second end 1042 of valve body 1036 and first side 1034 of base 1006. Valve gasket 1050 includes a front side surface 1071 configured to be disposed in sealing contact with second end 1042 of valve body 1036, and a back side surface (not visible in FIG. 10A) opposite from front side surface 1071 and configured to be in sealing contact with first side 1034 of base 1006. Valve body 1036 is rotatable relative to valve gasket 1050 about longitudinal axis 1046 of valve body 1036.

A tab 1072 on valve gasket 1050, which can also be referred to as a “key,” is configured to interlock and/or align with a corresponding recessed portion 1074, or keyway/groove, within bore 1030 of base 1006 to rotatably fix valve gasket 1050 relative to base 1006, such that valve assembly 1004 rotates relative to valve gasket 1050 which stays in place. The recessed portion 1074 is connected to first side 1034 and cylindrical inner wall 1032 of bore 1030. Passages 1076 (e.g., four shown) disposed through valve gasket 1050 correspond to ports 1052 (e.g., three shown) in lower wall 1020 of base 1006. Valve gasket 1050 is described in further detail with reference to FIGS. 10B-10C.

FIG. 10B illustrates a front side isometric view of valve gasket 1050 of FIG. 10A. Valve gasket 1050 is configured to form a seal between a valve assembly 1004 and housing 1005. Valve gasket 1050 comprises an outer portion 1070a, an inner portion 1070b, and a center portion 1070c which are each described in more detail herein.

Inner portion 1070b is comprised of a first elastomeric material that may be over-molded onto outer portion 1070a. In certain embodiments, the inner portion 1070b is formed from a silicone rubber or from a thermoplastic elastomer. The compressible nature of the first material of inner portion 1070b allows the thickness and the durometer of inner portion 1070b to be varied. In certain embodiments, inner portion 1070b may have a maximum diameter of, for example, 14.36 and a maximum height of, for example, 2.03 mm. Inner portion 1070b is circumscribed by outer portion 1070a, while itself circumscribing center portion 1070c, such that the diameter of inner portion 1070b is less than the diameter of outer portion 1070a and more than center portion 1070c. In an embodiment, an outer edge of inner portion 1070b is adjacent to outer portion 1070a, and an inner edge of inner portion 1070b is adjacent to center portion 1070c.

Passages 1076 (1076a-e) through inner portion 1070b are configured to facilitate fluid communication between housing 1005 and valve assembly 1004. Such passages 1076 may be partially defined by one or more spokes 1078 (1078a-e), or extensions, of inner portion 1070b, extending between center portion 1070c and outer portion 1070a. Although inner portion 1070b is shown as including five passages 1076, inner portion 1070b may include less than or more than five passages (e.g., 1-2 passages, 2-3 passages, 3-4 passages, 6-7 passages, or 7-8 passages).

Outer portion 1070a is comprised of a second, more rigid material that inner portion 1070b may be molded to. In certain embodiments, the second material of outer portion 1070a may be a thermoplastic substrate, such as polyamide or polycarbonate. In other embodiments, the second material of outer portion 1070a is molded from a thermoplastic substrate to which inner portion 1070b is mechanically overmolded or chemically bonded thereto. The second material of outer portion 1070a allows outer portion 1070a to be ultrasonically welded to base 1006. Outer portion 1070a may have a maximum diameter of 15.80 mm and a maximum height of 2.03 mm. Outer portion 1070a surrounds inner portion 1070b, such that the diameter of outer portion 1070a is greater than the diameter of inner portion 1070b and center portion 1070c. In an embodiment, an outer edge of outer portion 1070a is adjacent to cylindrical inner wall 1032 of bore 1030, and an inner edge of outer portion 1070a is adjacent to inner portion 1070b. Outer portion 1070a may be coupled to base 1006 of housing 1005 via ultrasonic welding, retaining ring 1038, a tab 1072, or a combination thereof.

Tab 1072 of outer portion 1070a is configured to constrain valve gasket 1050 within bore 1030 of base 1006. For example, tab 1072 constrains valve gasket 1050 by aligning with corresponding recessed portion 1074 of housing 1005 and preventing valve gasket 1050 from rotating when valve assembly 1004 is rotated within housing 1005. In certain embodiments, tab 1072 protrudes laterally from outer portion 1070a of valve gasket 1050 and is configured to align with corresponding recessed portions 1074 of housing 1005 to constrain valve gasket 1050 within housing 1005. In certain embodiments, tab 1072 recesses laterally from outer portion 1070a of valve gasket 1050 and is configured to align with a corresponding protruding portion of housing 1005 to constrain valve gasket 1050 within housing 1005. As an example, tab 1072 is a single rectangular tab. However, in some embodiments, outer portion 1070a may include more than one tab, which may protrude vertically, and which may be rectangular, circular, triangular, or any other suitable shape configured to restrain valve gasket 1050.

Center portion 1070c is comprised of a third material that may be different from, or the same as, the first and second materials of the inner and outer portions 1070b, 1070a. As an example, the third material of center portion 1070c may be the same material as outer portion 1070a, such as a thermoplastic, or may be other rigid material. Center portion 1070c may have a maximum diameter of, for example, 3.80 mm and a minimum height of, for example, 3.05 mm. Center portion 1070c is surrounded by inner portion 1070b along an outer edge, such that the diameter of center portion 1070c is less than the diameter of inner portion 1070b and outer portion 1070a.

In some embodiments, center portion 1070c is configured to align valve gasket 1050 with valve assembly 1004 and housing 1005. For example, center portion 1070c is configured to align valve gasket 1050 along longitudinal axis 1046 with valve assembly 1004 and housing 1005. In certain embodiments, center portion 1070c is connected to outer portion 1070a, such as through inner portion 1070b. In such examples, center portion 1070c and outer portion 1070a may be monolithically formed, and inner portion 1070b over-molded thereon, such that center portion 1070c may be connected to outer portion 1070a through spokes 1078 of inner portion 1070b.

In the embodiment shown by FIG. 10B, center portion 1070c is configured as a key, or alignment feature, for aligning valve gasket 1050 with valve assembly 1004 and housing 1005. As an example, center portion 1070c may include a protruding portion 1080 (e.g., a protruding ring) that protrudes from front side surface 1071 and that is configured to align with a recessed portion (e.g., a recessed ring) of valve assembly 1004. Alternatively, the protruding portion 1080 may protrude from back side surface of valve gasket 1050, such that the protruding portion is configured to align with a recessed portion of first side 1034 of base 1006. Center portion 1070c may also include a cavity or may be omitted entirely (i.e., removed from valve gasket 1050), such that a protruding portion of valve assembly 1004 or housing 1005 is configured to align with the cavity or the omitted center portion 1070c for alignment of the valve assembly 1004.

Manufacturing valve gasket 1050 may involve a two-shot or over-molding manufacturing process that forms one gasket structure. In some embodiments, manufacturing valve gasket 1050 comprises over-molding inner portion 1070b from a first material over outer portion 1070a, where outer portion 1070a is molded from a second material before the over-molding of inner portion 1070b. Center portion 1070c may optionally be molded from the second material at the same time as outer portion 1070a, or may be over-molded from a third material over inner portion 1070b at a same or different time as inner portion 1070b.

Greater detail of the interaction between valve gasket 1050 and first side 1034 of base 1006 may be had in FIG. 10C. Valve assembly 1004 and base 1006 of FIG. 10C may be as described with reference to FIG. 5A unless otherwise expressly noted. Specifically, when viewed in cross-section, valve gasket 1050 forms a seal between valve assembly 1004 and base 1006 of housing 1005. Because the second material of outer portion 1070a of valve gasket 1050 may be a plastic substrate, outer portion 1070a may be ultrasonically welded to housing base 1006. Application of appropriate surface finishes to inner portion 1070b, in conjunction with the first material of inner portion 1070b, may reduce potential for resistance causing abrasion or increased torque. Additionally, use of valve gasket 1050 allows valve assembly 1004 to form a seal without a valve elastomer (e.g., valve elastomer 251 or 451). By replacing the valve elastomer with valve gasket 1050, resistance during valve rotation may be reduced because the valve elastomer may not balloon into ports 1052 during rotation of valve assembly 1004.

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein but are to be accorded the full scope consistent with the language of the claims.

### Example Embodiments

Embodiment 1: A surgical cassette, comprising: a housing; a retaining ring coupled to the housing; a valve assembly coupled to the housing by the retaining ring and configured to control fluid communication of a channel within the housing; and a valve gasket configured to form a seal between the valve assembly and the housing, the valve gasket comprising: an inner portion comprised of a first material; and an outer portion comprised of a second material, wherein the outer portion surrounds the inner portion and is coupled to the housing.

Embodiment 2: The surgical cassette of Embodiment 1, wherein: the first material of the inner portion is molded from an elastomeric material; and the second material of the outer portion is molded from a thermoplastic substrate to which the inner portion is mechanically overmolded or chemically bonded thereto.

Embodiment 3: The surgical cassette of Embodiment 2, wherein a surface of the inner portion is coated with a material configured to decrease a coefficient of friction of the valve gasket.

Embodiment 4: The surgical cassette of Embodiment 2, wherein the first material is an elastomeric material containing a lubricant configured to decrease a coefficient of friction of the valve gasket.

Embodiment 5: The surgical cassette of Embodiment 1, wherein a passage through the inner portion of the valve gasket is configured to facilitate fluid communication between the housing and the valve assembly.

Embodiment 6: The surgical cassette of Embodiment 1, wherein the outer portion is ultrasonically welded to the housing to constrain the valve gasket in the housing.

Embodiment 7: The surgical cassette of Embodiment 1, wherein the valve assembly rotates relative to the valve gasket.

Embodiment 8: A surgical cassette, comprising: a housing; a retaining ring coupled to the housing; a valve assembly coupled to the housing by the retaining ring and configured to control fluid communication of a channel within the housing; and a valve gasket configured to form a seal between the valve assembly and the housing, the valve gasket comprising: an inner portion comprised of a first material; and an outer portion comprised of a second material, wherein the outer portion surrounds the inner portion, is coupled to the housing, and comprises a tab configured to constrain the valve gasket within the housing.

Embodiment 9: The surgical cassette of Embodiment 8, wherein the tab: is configured to align with a recessed portion of the housing to constrain the valve gasket within the housing; and protrudes laterally from the outer portion of the valve gasket.

Embodiment 10: The surgical cassette of Embodiment 8, wherein the tab: is configured to align with a protruding portion of the housing to constrain the valve gasket within the housing; and recesses laterally from the outer portion of the valve gasket.

Embodiment 11: A surgical cassette, comprising: a housing; a retaining ring coupled to the housing; a valve assembly coupled to the housing by the retaining ring and configured to control fluid communication of a channel within the housing; and a valve gasket configured to form a seal between the valve assembly and the housing, the valve gasket comprising: an inner portion comprised of a first material; an outer portion comprised of a second material that surrounds the inner portion; and a center portion surrounded by the inner portion and configured to align the valve gasket with the valve assembly and the housing.

Embodiment 12: The surgical cassette of Embodiment 11, wherein the center portion comprises a cavity that is configured to align with a protruding portion of the valve assembly or a protruding portion of the housing.

Embodiment 13: The surgical cassette of Embodiment 11, wherein the center portion comprises a key configured to align the valve gasket with the valve assembly and the housing.

Embodiment 14: The surgical cassette of Embodiment 13, wherein the key of the center portion comprises a protruding portion that is configured to align with a recessed portion of the valve assembly or a recessed portion of the housing.

Embodiment 15: The surgical cassette of Embodiment 13, wherein the key of the center portion comprises a protruding ring that is configured to align with a recessed ring of the valve assembly or a recessed ring of the housing.

What is claimed is:

1. A surgical cassette, comprising:
a housing;
a retaining ring coupled to the housing; and
a valve assembly coupled to the housing by the retaining ring and configured to control fluid communication of a channel within the housing, the valve assembly comprising:
a valve body having a first end, a second end, and a cylindrical surface connecting the first end and the second end; and
a valve elastomer comprising:
a first side configured to couple the valve elastomer to the second end of the valve body; and
a second side having a recessed surface,
wherein the recessed surface is configured to:
engage with the housing to form a seal with a base of the housing, and
reduce mechanical stress across the valve elastomer during rotation of the valve assembly.

2. The surgical cassette of claim 1, wherein the recessed surface of the second side of the valve elastomer comprises a first semi-circular inner edge, two straight sides from the first semi-circular inner edge to an area of largest width of the recessed surface, and a second semi-circular outer edge.

3. The surgical cassette of claim 1, wherein a protruding portion of the first side of the valve elastomer is configured to engage with a corresponding recessed portion of the second end of the valve body to couple the valve elastomer to the valve body.

4. The surgical cassette of claim 1, wherein the valve elastomer is coated with a self-lubricating material.

5. A surgical cassette, comprising:
a housing for receiving a valve assembly, the housing comprising:
a base having a first side configured to contact the valve assembly, the base comprising:
a port through the base configured to facilitate fluid communication of a channel within the housing; and
a recessed surface formed in the first side,
wherein the recessed surface is configured to:
engage with the valve assembly to form a seal with a valve elastomer of the valve assembly, and
reduce mechanical stress across the valve elastomer during rotation of the valve assembly.

6. The surgical cassette of claim 5, wherein the recessed surface of the first side comprises a first semi-circular inner edge, two straight sides from the first semi-circular inner edge to an area of largest width of the recessed surface, and a second semi-circular outer edge.

7. The surgical cassette of claim 5, wherein the recessed surface of the first side is adjacent to the port of the base.

8. The surgical cassette of claim 5, wherein the port comprises a ring protruding from the first side around a perimeter of the port.

9. A surgical cassette, comprising:
a housing for receiving a valve assembly, the housing comprising:
a base having a first side configured to contact the valve assembly, the base comprising:
a port through the base configured to facilitate fluid communication of a channel within the housing,
wherein the port comprises a ring protruding from the first side around a perimeter of the port, and
wherein the ring protruding from the first side is configured to:
engage with the valve assembly to form a seal with the valve assembly, and
prevent a liquid from leaking into the port during rotation of the valve assembly.

10. The surgical cassette of claim 9, wherein the protruding ring is configured to form the seal between the valve assembly and the housing.

11. The surgical cassette of claim 9, wherein the protruding ring is configured to engage with the valve assembly to compress an elastomeric sealing material against the first side of the base.

12. The surgical cassette of claim 9, wherein the protruding ring comprises a first semi-circular inner edge, two straight sides from the first semi-circular inner edge to an area of largest width of the ring, and a second semi-circular outer edge.

13. The surgical cassette of claim 9, wherein the protruding ring is configured to provide localized sealing around the port.

14. The surgical cassette of claim 9, wherein the protruding ring is configured to align with a recessed portion of the valve assembly.

15. The surgical cassette of claim 9, wherein the protruding ring is configured to prevent the liquid from leaking into the port during the rotation of the valve assembly by squeegeeing a valve elastomer of the valve assembly.
